(19) United States
(12) Patent Application Publication
(10) Pub. No.: US 2003/0077635 A1
(43) Pub. Date: Apr. 24, 2003

(54) Dendrimers and methods for their preparation and use

(75) Inventor: Jesper Lohse, Copenhagen NV (DK)

Correspondence Address: JACOBSON HOLMAN PLLC, 400 SEVENTH STREET N.W., SUITE 600, WASHINGTON, DC, 20004, US

(73) Assignee: DAKO A/S

(21) Appl. No.: 10/238,732

(22) Filed: Sep. 11, 2002

Related U.S. Application Data
(63) Continuation of application No. 09/606,315, filed on Jun. 29, 2000, now abandoned.

(30) Foreign Application Priority Data
Jun. 29, 1999 (DK) PA 1999 00934

(51) Int. Cl. C12Q 1/68; G01N 33/53; G01N 33/537; G01N 33/543; C08G 63/48; C08G 63/91

(52) U.S. Cl. 435/6; 435/7.5; 435/7.92; 525/54.5

(57) ABSTRACT

Novel dendrimers as well as novel dendrimer complexes are disclosed. Such dendrimers and/or dendrimer complexes may be used for the detection of various components of a sample and as detection systems and signal enhancement/amplification systems. The dendrimers and dendrimer complexes may also be used for labelling various entities/compounds. Furthermore, labelling kits and detection kits comprising one or more labelled dendrimers or one or more dendrimer complexes are also one of the possible uses.

### FIELD OF THE INVENTION

The present invention relates to the field of polymer chemistry and, in particular to novel dendrimers as well as novel dendrimer complexes. In a further aspect, the invention concerns fields of use for such dendrimers and/or dendrimer complexes in the detection of various components of a sample, as detection systems, signal enhancement/amplification systems, and labelling systems. Furthermore, kits comprising the dendrimers/dendrimer complexes are also part of the present invention.

### BACKGROUND OF THE INVENTION

Probes that allow for the detection of specific biological markers such as proteins and nucleic acids are of wide-spread use and are a valuable tool for both in vitro and in vivo diagnostic purposes as well as in life-science research. However, detection of these markers can be difficult as not all are present in a sufficiently high number. This makes the detection of low copy number targets with a single labelled probe impossible or impractical even though the probe may hybridise to the target with a high degree of specificity and fidelity. This substantial limitation has facilitated the development of various detection systems, the purpose of which is to enhance the signal that is detectable in an assay. Examples of such systems include both target amplification (e.g. PCR, NASBA, SDA and LCR) as well as signal amplification techniques (e.g. Tyramide Signal Amplification).

Despite of the many different detection systems available, there still exists an opportunity for improvement. Dendrimers presents one such opportunity to improve detectable signal in assays employing targets of low copy number because single probes can be modified with multiple detectable labels as a means to increase signal output from the probe.

In general, dendrimers are well-defined macromolecules that diverge from a focal point through multiple branching points to terminate in multiple peripheral functional groups. These peripheral functional groups can be used to link multiple labels (e.g. biotin, fluorophores, or combinations thereof) to other molecules such as DNA oligomers. Alternatively, multiple macromolecules such as peptides or nucleic acids, or a combination of the two, can be linked to the dendrimers. The ability to link the same or different molecules of choice at the periphery of the dendrimer provides for signal amplification potential.

For example, dendrimers have been used to improve an immune response (e.g. in vaccination strategies) by facilitating the presentation of antigens or antigenic peptides to the immune system. Specifically, Multiple Antigenic Peptides (MAPs) and Antigen Presenting Cells (APCs) are related systems where multiple peptides are synthesised or grafted upon a small polylysine core in order to enhance the antigenic properties relative to the individual peptides.

The macromolecules applied to dendrimer formation typically included linear macromolecules (e.g. dextran), branched macromolecules (e.g. hydrolysed starch), cyclic macromolecules (e.g. cyclodextrans) and spherical macromolecules (e.g. nanoparticles). They have in particular been used to display multiple probes such as antibodies, or signal generating proteins such as HRP, or combinations thereof.

Notwithstanding their prevalent use, the known dendrimers have several drawbacks and limitations. For example, most of the known dendrimers are very compact structures giving raise to significant steric hindrance or crowding in the outer layers (periphery) of the dendrimer. This is especially pronounced in the case of MAPS, since there are only 3 or 6 bonds between branching points. The well known StarBurst™ dendrimers comprise only 7 bonds between branching points and therefore soon reach a situation where there is little or no free space available for conjugation at the periphery of the dendrimer; even for such small molecules (labels) such as biotin (ref. 1). With such little available space at the periphery, larger molecules such as DNA oligomers are particularly difficult to conjugate to the dendrimer; and if successfully conjugated, the kinetics of hybridisation to the oligomer are limited by the crowded periphery of the macromolecular complex.

For example, dendrimers of DNA oligomers have been prepared. These are, however, very large and bulky molecules. While the spacing between branching points can be several hundred bonds, the double helices that hold the structure together are very bulky relative to their longitudinal extension. Large bulky molecules can also be difficult to work with in certain applications since they can exhibit reduced cellular uptake and mobility.

Yet another limitation of conventional dendrimers is that they typically must be prepared by solid phase chemistry if the impurities are to be successfully removed. In particular, MAPs are almost always prepared by solid phase techniques, thus, limiting this technique to smaller linear peptides as larger biomulecules in general are incompatible with the harsh cleavage/deprotection conditions.

Whilst still another drawback of conventional dendrimers is that the vast majority of dendrimers are homo-functional. Because they are homofucntional, it can be difficult or impossible to attach different ligand to the dendrimer is a well-defined manner.

Because of the crowing, specific types of signal amplication can be difficult to achieve in conventional dendrimers. For example, when multiple (identical or different) fluorophores are linked to dendrimers having a crowded periphery, the fluorophores are likely to quench each other by collision induced or by quantum transfer of energy or electrons where the efficiency of quenching is proportional to the sixth power of distance between the fluorophores. This quenching limits signal output since the large number of fluorophores produce a signal that is significantly less than the sum of the signal possible from each of the individual fluorphores and thereby thwarts the concept of linking multiple fluorophores to a dendrimer to thereby achieve substantial improvement in signal output from a single species (e.g. a DNA probe).

Background straining is another well-known problem in detection. In the field of multi-layer visualisation systems, the potential background problem increases with the number of layers and their chemical diversity. Blocking and stringent washing conditions must be optimised for each layer, however, still be compatible with prior and subsequent layers, thus, limiting the construction of multi-layer systems.

### SUMMARY OF THE INVENTION

By the present invention, novel dendrimers and dendrimer complexes are provided which do not possess the above-mentioned drawbacks and limitations. The dendrimers and dendrimer complexes of the invention provide spacing between the terminal peripheral functional groups to which various compounds are attached. Thus, the dendrimers and dendrimer complexes of the present invention have a less compact or crowded structure, particularly at the periphery, as compared with the known dendrimers. Indeed, the dendrimers of the present invention are of low molecular weight as compared to their spatial size and are therefore less bulky as compared with conventional dendrimers such as MAPs and APCs. Furthermore, the dendrimers of the present invention can be prepared by liquid phase chemistry.

Finally, and of significant importance, the dendrimers of the present invention are well-defined in molecular structure and are heteromulitfunctional, i.e. the dendrimer/dendrimer complex enable at least two different substituents/entities, and further enable presentation of at least one of these in multiple copies. Consequently, many combinations of different moieties can be linked to the periphery of the dendrimers of the present invention is a highly defined manner and with a highly defined stoichometry. Thus, the dendrimers of the present invention can be tailor made for specific applications and will not suffer from lot to lot variations commonly observed with other polymeric conjugates. Therefore, the dendrimers and dendrimer complexes of the present invention are useful in a wide range of applications, e.g. as detection systems and signal enhancement systems.

In a first aspect, the present invention relates to novel dendrimers having the general formula (I)

(Entity1)ₓ₁-A-(Entity2)ₓ₂ (I)

wherein A is a dendritic core having at least one N-atomic branching point, said branching point not being part of a naturally occurring amino acid, and wherein A comprises at least one ether group,

each Entity1 and Entity2 are independently a probe, a probe substituted by one or more labelling compounds, a labelling compound, or a probe having reactive groups,

x1 and x2 are independently 0 or an integer of from 1 to 1200,

and protected forms thereof.

In another aspect, the present invention relates to novel dendrimer complexes and protected forms thereof comprising at least one dendrimer as defined herein, which dendrimer or dendrimers are connected to other compounds of interest, e.g. a naturally or non-naturally amino acid terminally or internally, a peptide nucleic acid terminally or internally, an LNA terminally or internally, a peptide terminally or internally, a protein terminally or internally, an antibody, an antigen, an immune complex, a RNA sequence or an analogue thereof terminally or internally, a DNA sequence or an analogue terminally or internally, a macromolecule terminally or internally, or a solid or semi-solid support.

The dendrimers and dendrimer complexes as well as the protected forms thereof of the present invention have a broad range of possible uses including the use in the detection of the presence of various components of a sample, like the detection of nucleic acid sequences, antibodies, antigens, immune complexes, proteins, or peptides.

Furthermore, the dendrimers and dendrimer complexes of the present invention may be used as or in detection systems and signal amplification systems. Such systems may suitably be used in the detection of various components of a sample, including the detection of nucleic acid sequences, antibodies, antigens, immune complexes, proteins, or peptides.

Also, the dendrimers and dendrimer complexes of the present invention enable the application in in vitro and in vivo diagnostic methods. Accordingly, the present invention i.a. relates to detection kits and amplification kits comprising such dendrimers and dendrimer complexes and/or protected forms thereof.

Another possible use of the dendrimers and dendrimer complexes as well as the protected forms thereof of the present invention is in the labelling of various compounds. Thus, the present invention further relates to the use of the dendrimers and dendrimer complexes as well as the protected forms thereof in labelling reactions as well as to labelling kits comprising such dendrimers and/or dendrimer complexes, and/or protected forms thereof.

The present invention is described in detail in the following.

### BRIEF DESCRIPTION OF THE FIGURES

FIG. 1 shows examples of various suitable protecting groups.

FIG. 2 shows examples of activated groups.

FIG. 3 shows examples of free groups.

FIGS. 4-17 shows the various dendrimers of the Examples.

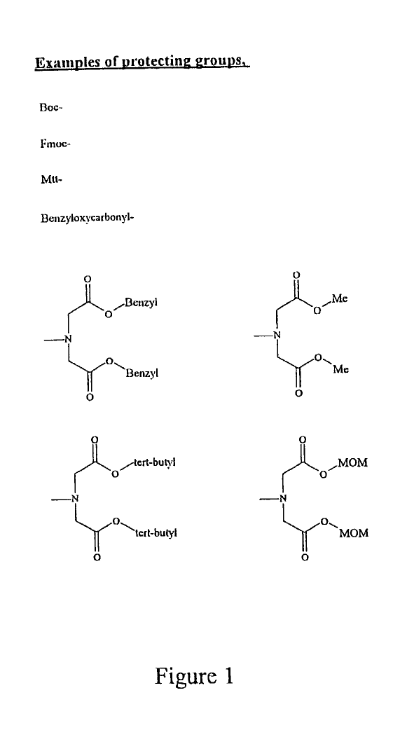

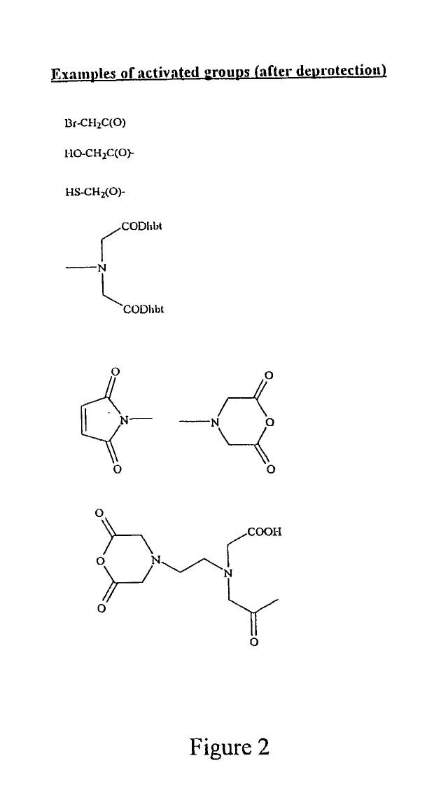

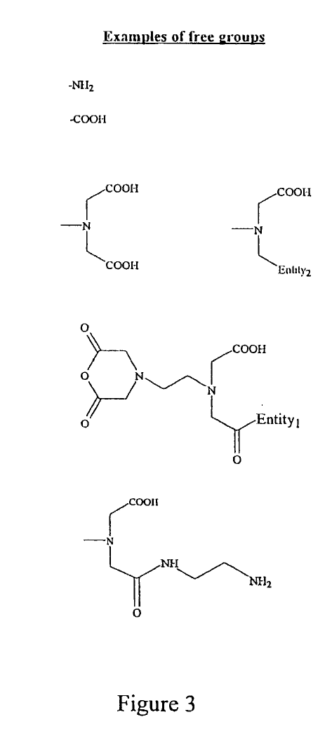

### DETAILED DESCRIPTION OF THE INVENTION

In a first aspect, the present invention relates to dendrimers having the general formula (I)

(Entity1)ₓ₁-A-(Entity2)ₓ₂ (I)

wherein A is a dendritic core having at least one N-atomic branching point, said branching point not being part of a naturally occurring amino acid, and wherein A comprises at least one ether group,

each Entity1 and Entity2 are independently a probe, a probe substituted by one or more labelling compounds, a labelling compound, or a probe having reactive groups,

x1 and x2 are independently 0 or an integer of from 1 to 1200,

and protected forms thereof.

From WO 98/32469 (ref. 2), compounds comprising a linear, branched or dendrimeric polymer backbone comprising a plurality of amine-containing acids are known. In particular, the polymeric backbone is composed of native or non-native amino acid residues. Such compounds have linked thereto at least one reporter moiety. The polymers (dendrimers) may be asymmetrical or symmetrical. The dendrimers are useful as therapeutic and diagnostic agents. The dendrimers of the present invention differ from the dendrimers disclosed in WO 98/32469 in that the dendritic core has at least one N-atomic branching point that is not part of a naturally occurring amino acid, and further in that the dendritic core comprises at least one ether group.

From EP 271 180 (ref. 3), and Bioconjugate Chem. 9, 813-825 (1998) (ref. 4), so-called StarBurst™ dendrimers are known. Such dendrimers are dense structures exhibiting a molecular architecture characterised by regular dendritic branching with radial symmetry. The StartBurst™ dendrimers are suitable for carrying various materials. The dendrimers of the present invention differ from the dendrimers disclosed in EP 271 180 (ref. 3) and Bioconjugate Chem. (ref. 4) in that the dendritic core comprises at least one ether group, and further in that the dendrimers of the present invention are heteromultifunctional, whereas all peripheral groups of the conventional StarBurst™ dendrimers are identical.

From WO 97/07398 (ref. 5), dendrimer/polypeptide complexes are known. The complex comprises a dendrimer with a plurality of termini, having coupled thereto a first and a second polypeptide, whereby the formed complex exhibits first and second defined biological activities. Also comprised is dendrimer/polypeptide complexes, wherein a first and a second are coupled to each other. The dendrimers to be used in accordance with WO 97/07398 (ref. 5) are not defined specifically as the invention as defined in this document does not seem to lie in the dendrimers as such. However, from the description, it appears that the StarBurst™ dendrimers are suitable. As mentioned above, the dendrimers of the present invention differ from the StarBurst™ dendrimers in that the dendritic core comprises at least one ether group, and further in that the dendrimers of the present invention are heteromultifunctional, whereas all peripheral groups of the conventional StarBurst™ dendrimers are identical.

The StarBurst™ dendrimers are dense structures exhibiting “starburst dense packing”, where the surface of the dendrimer contains sufficient terminal moieties such that the dendrimer surface becomes congested and encloses void spaces within the interior of the dendrimers, and this congestion can provide a molecular level barrier which can be used to control diffusion of material into or out of the interior of the dendrimer, cf. page 6, lines 11-19 of EP 271 180 (ref. 3). This so-called “starburst topology” is achieved by assembling organic repeating units in concentric, dendritic tiers around an initiator core, cf. page 2, lines 20-31 of EP 271 180 (ref. 3). The topology is achieved by forming successive layers of relatively short amino-containing chain units, in particular the PEI and PAMAM, cf. 31 and the examples of EP 271 180 (ref. 3). As mentioned, this leads to very dense structures, and the materials to be carried may have to carry connectors and/or spacers to facilitate the use of the starburst conjugates, cf. page 13, line 29 to page 14, line 1 of EP 271 180 (ref. 3).

In WO 98/32469 (ref. 2), it is mentioned that the invention lies in the recognition that co-polymers of amino acids carrying or attached to one or more reporter groups are particularly suitable for diagnostic and therapeutic use, cf. page 4, third paragraph. As mentioned on page 6, second paragraph of WO 98/32469 (ref. 2), the dendrimers comprise a plurality of native or non-native amino acids. It can be derived from page 7, third paragraph of WO 98/32469 (ref. 2) that the use of amino acids results in that the dendrimer becomes biodegradable. The dendrimer may also comprise other moieties, cf. page 6, third paragraph of WO 98/32469 (ref. 2). Such other moieties are relatively short amino-containing chains. The use of successive layer of amino acids and/or other moieties results in dendrimers with a very dense structure.

As mentioned in the introductory part of this description, such dense dendrimer structures have several drawbacks, i.a. significant steric hindrance and crowding in the outer layer of the dendrimer, and quenching of fluorphores attached to the outer layers of the dendrimer. In contrast hereto, the dendrimers and dendrimer complexes of the present invention do not possess these drawbacks. Thus, the advantages of the dendrimers and dendrimer complexes of the present invention are numerous. They have a sufficiently loose structure to allow conjugation of even quite large entities. Furthermore, the closest neighbouring anchoring groups are sufficiently far apart, whereby reduced reactivity, aggregation of the attached entities, fluorescence quenching and other undesirable effects of steric crowding are avoided or minimised. Furthermore, another pronounced advantage of the dendrimers and dendrimer complexes of the present invention is that they generally have good solubility due to a content of hydrophilic groups. Also, the dendrimers are easily derivatised with a desired entity, e.g. a probe or a labelling compound, whereby the full potential of the multiple sites can be fully exploited, and well-defined conjugates prepared. Furthermore, it is possible to activate the dendrimer in advance, so that chemical modifications of the attached entities are avoided, and naturally occurring functionalities such as amines, carboxylic acids, thiols, alcohols etc. can be brought to react spontaneously with the dendrimer. In particular, the dendrimer may be heterofunctional. In this way, one type of entity, e.g. a probe or a labelling compound, can be attached to one or some groups on the dendrimer, while other entities, e.g. other probes or labelling compounds, can be attached to other groups using orthogonal chemistries, the exact stochiometry being controlled by different chemistries, not by having different entities competing for the same sites. For example, dendrimers having 17 bonds between branching points can be constructed wherein there at the periphery are 40 bonds between nearest neighbouring anchoring groups. The dendrimers can e.g. be derivatised as a mono Boc-amino polyanhydride, or polyBoc-amino mono anhydride. An advantage of the present dendrimers is their easy derivatisation with peripheral imodiacetic acid anhydride moieties. This moiety reacts very fast and selectively with primary aliphatic amines allowing conjugation in water under aqueous conditions with unprotected nucleic acids or analogues thereof such as peptide nucleic acids. Conversely, the Boc group can be removed and the resulting free amino group can be reacted directly with activated carboxylic acids, can be reacted to yield malimides or 2-haloacetyls that are thiol reactive, or can be reacted to give an anhydride that again is amino-reactive. Beneficial is the complete orthogonality obtained between multiple benzyl ester groups, and a Boc-amino group. Thus, herterofunctional dendrimers can readily be prepared.

In the dendrimers of the present invention, the dendritic core A extends from a single focal point through multiple generations of successive layers, each layer having one or more branching points, having regard to the definitions above.

In particular, the dendritic core A comprises one or more of C₁₋₁₀₀ alkyl groups, C₂₋₁₀₀ alkenyl groups, C₂₋₁₀₀ alkynyl groups, said alkyl, alkenyl, and alkynyl groups optionally containing one or more functional groups and/or one or more heteroatoms, naturally or non-naturally amino acids, peptide nucleic acid moieties, LNAs, peptides, proteins, antibodies, antigens, immune complexes, DNA sequences or analogues thereof, RNA sequences or analogues thereof, macromolecules, and solid or semi-solid supports, however, having regard to the provisos above, i.e. that the dendritic core has at least one N-atomic branching point, said branching point not being part of a naturally occurring amino acid, and wherein A comprises at least one ether group. It is to be understood that each layer may comprise one or more of the residues mentioned.

The alkyl, alkenyl and alkynyl groups may in particular be such with C₅₋₈₀, C₅₋₇₀, C₅₋₆₀, C₁₀₋₅₀, C₁₀₋₂₅, C₁₅₋₂₅, and C₁₀₋₂₀ carbon atoms which further optionally contains one or more functional groups and/or one or more heteroatoms. The alkyl, alkenyl and groups may be straight chain or branched groups.

The term “functional group” is intended to comprise groups such as e.g. ester groups, ether groups, thiol groups, carbonyl groups, hydroxyl groups, amide groups, carboxylic groups, and imide groups as well as combinations thereof.

The term “heteroatoms” is intended to include i.a. O, N, S and P.

Within the present context, the term “probe” is intended to mean a compound of chemical or biological origin that specifically recognises and binds to markers and/or complexes thereof. Several probes can be envisaged. Ways of selecting probes targeting a desired marker or nucleic acid sequence are known to the person skilled in the art. Suitable probe sequences include i.a. such originating from bacteria, viruses, fungi, allergens, chromosome and markers. It is within the scope of the present that several probes may be connected to each other. Thus, the expression “a probe” is intended to include a combination of probes which may be connected to each other terminally or internally.

The terms “RNA” and “DNA” are of course well known expressions and it is accordingly believed that no specific references need to be mentioned. The term “DNA” further comprise both the sense and the anti-sense strands. Analogues of RNA and DNA is intended to comprise any chemical modification of such, e.g. modified linkages (like phosphothioates), modified riboses (like LNA) or modified bases (e.g. 5-substituted uracils). “Peptide nucleic acids” are synthetic molecules in some aspects resembling the functions of DNA. Peptide nucleic acids were firstly disclosed in WO 92/20702 (ref. 6) and Wo 92/20703 (ref. 7). The term “peptide nucleic acid” is to be interpreted broadly as it conventionally used to characterise compounds having a polymeric backbone, cf. the definition of WO 92/20702 (ref. 6) and WO 92/20703 (ref. 7). “LNAs” are newly invented compounds also in some aspects resembling the functions of DNA. “Peptides”, “proteins”, “antibodies”, “antigens” and “immune complexes” are well known terms, and is within the present context intended to include any such suitable for the purposes of the present invention. The antibodies and immune complexes may be used as monomers, dimers, trimers, tetramers, or various multimers/aggregates. Also included are antibodies/immune complexes of from the classes IgA, IgD, IgE, IgG and IgM as well as subclasses thereof. The term “antibody” also comprises epitopes, and various fragments of such antibodies, e.g. Fab fragments and single-chain fragments. Also comprised are monoclonal, polyclonal, and recombinant antibodies. The term “macromolecules” is intended to include dextrans, polyvinylpyrrolidone, branched or linear polyethers, and branched or linear polylysines. Within the present context, the term “solid or semi-solid support” is intended to include particles and beads e.g. of polystyrenes, polypropylenes, polyethylenes, dextrans, nylon, amyloses, celluloses, polyacrylamides and agarose, membranes e.g. of cellulose, cellulose acetate, glycerols and polyvinylidene fluoride.

Both the dendrimers as well as the protected forms of such dendrimers are aspects of the present invention. A protected form is such having protecting groups attached thereof. The dendritic core itself may be protected by protecting groups (e.g. in the case where x1 or x2, or x1 and x2 are 0). Such protecting groups may be located at the outermost layer of the dendritic core. Also, Entity1 and Entity2 may independently be

a protecting group,

a probe protected by a protecting group (a protected probe),

a labelling compound protected by a protecting group (a protected labelling compound),

a protected probe substituted by one or more labelling compounds,

a protected probe substituted by one or more protected labelling compounds,

a probe having reactive group protected by a protecting group (a protected reactive group).

Examples of suitable protecting groups are Fmoc, Boc, Mtt, Mmt, Dde, All, Aloc, ODmab, OtBu, Ome, Obz, Z, MOM and benzyloxycarbonyl.

The dendritic core may terminate in a free group, a reactive group or an activated group (e.g. when x1 or x2 or x1 and x2 are 0). A few examples are amines and acids.

In a preferred embodiment, the dendritic core A may in each consecutive layer comprise one or more of

—NH((CH₂)_{q}O))_{q}(CH₂)_{q}N((CH₂)_{q}C(O)—)₂,

((—CH₂)_{q}O)_{q}(CH₂)_{q}N((CH₂)_{q}O—)₂,

((—CH₂)_{q}O)_{q}(CH₂)_{q}C((CH₂)_{q}O—)₃, and

—NH((CH₂)_{q}O)_{q}(CH₂)_{q}C((CH₂)_{q}C(O)—)₃,

wherein each q independently is 0 an integer from 1 to 8, preferably from 1 to 3. Also comprised is successive layers containing one of more of these specified groups as well as one or more of naturally or non-naturally amino acids, peptide nucleic acid moieties, LNAs, peptides, proteins, antibodies, antigens, immune complexes, DNA sequences or analogues thereof, RNA sequences or analogues thereof, macromolecules, and solid or semi-solid supports, and further layers containing one or more of these specified groups and other layers containing one or more of naturally or non-naturally amino acids, peptide nucleic acid moieties, LNAs, peptides, proteins, antibodies, antigens, immune complexes, DNA sequences or analogues thereof, RNA sequences or analogues thereof, macromolecules, and solid or semi-solid supports.

Suitable components further include such groups which have a hydrophilic character. Zwitterionic groups are also comprised.

In particular, the dendritic core A may comprises one or more of

—NH(CH₂)₃O(CH₂)₂O(CH₂)₂O(CH₂)₃N(CH₂C(O))₂—,

—NH(CH₂)₂NHC(O)CH₂(N(CH₂CH₂)N)CH₂C(O)—

(—NH(CH₂)₃(O)(CH₂)₂O(CH₂)₂O(CH₂)₃N(CH₂COOH)CH₂C(O))_{q}, wherein q is as defined above,

—NH(CH₂)₃O(CH₂)₂O(CH₂)₂O(CH₂)₃N(COOH)CH₂C(O)—

—NH(CH₃)₃(OCH₂CH₂)_{q}O(CH₂)₃N(CH₂COOH)CH₂C(O)—

—NH(CH₂)₃O(CH₂)₂O(CH₂)₂O(CH₂)₃NHC(O)CH₂O(CH₂CH₂)_{q}CH₂C(O)NH—(CH₂)₃O(CH₂)₂O(CH₂)₂O(CH₂)₃N(CH₂COOH)CH₂C(O)—.

In a particular embodiment, the dendritic core comprise one or more of the layer compounds ALL, DAAL, TAAL, PEG, PEG34, and PIP cf. the abbreviations below.

In certain applications, it may be advantageous that the dendritic core A is composed of identical units forming successive layers having the same basic structure. In orther, it may be advantageous that the dendritic core A is composed of different units forming layers having an alternating structure.

Each layer of the dendritic core A may suitably be bifocated or trifocated. By this is meant that each layer has two or three possible branching points. Two branching points can be provided by trivalent nitrogen atoms, and three branching points can be provided by tetravalent carbon atoms.

In accordance with the definition above, the dendritic core A comprises at least one N-atomic branching point, said branching point not being part of a naturally occurring amino acid, and wherein A comprises at least one ether group. Thus, the dendrimer is not composed entirely of naturally occurring amino acids. However, in some applications, it may be an advantage that the dendritic core A comprises naturally or non-naturally occurring amino acids.

The loose structure of the dendrimers of the present invention can be achieved by forming a dendritic core A such that the interdistance between each of the Entity1's and/or each of the Entity2's or visa versa is at least 5, at least 10, at least 20, at least 30, at least 35, at least 40, at least 50, at least 60, at least 70, at least 80, at least 90, at least 100, at least 110, at least 120, at least 130, at least 140, at least 150, at least 200, at least 210, at least 220, at least 230, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 525, at least 550, at least 575, or at least 600 bonds. In some embodiments, the interdistance is 35, 770, 130 and 250 bonds, cf. the Examples. By the expression “interdistance” is meant the number of bonds between two entities being attached to same branching point, however, without bonds to other branchings.

As appears from the above, the present invention relates to dendrimers, wherein

x1 and x2 are both 0,

x1 is 0, and x2 is 1-1200,

x1 is 1-1200, and x2 is 0, and

x1 is 1-1200, and x2 is 1-1200.

The dendrimers of the present invention can be constructed so as to present any combination of Entity1's and Entity2's. Particular interesting possibilities are

(1) the Entity1's are probes and the Entity2's are labelling compounds,

(2) the Entity1's are labelling compounds and the Entity2's are probes,

(3) both the Entity1's and the Entity2's are probes,

(4) both the Entity1's and the Entity2's are labelling compounds,

(5) the Entity1's are probes substituted by labelling compounds, and the Entity2's are labelling compounds,

(6) the Entity1's are labelling compounds, and the Entity2's are probes substituted by labelling compound,

(7) one or more of the Entity1's are probes, and the remaining Entity1's are another of the possible substituents as defined above, and the Entity2's are labelling compounds or probes substituted by labelling compounds,

(8) one or more of the Entity2's are probes, and the remaining Entity2's are another of the possible substituents as defined above, and the Entity1's are labelling compounds or probes substituted by labelling compounds,

(9) the Entity1's are the same or different labelling compounds or the same or different probes substituted by the same or different labelling compounds,

(10) the Entity2's are the same or different labelling compounds or the same or different probes substituted by the same or different labelling compounds,

(11) only Entity1's are present (x1 being 0),

(12) only Entity2's are present (x2 being 0), as well as protected forms of such dendrimers.

As clearly indicated, the dendrimers of the present invention enable multi-colour presentation (or multi-signal presentation) by choosing the probes/labelling compounds of all or only some the Entity1's and/or all or only some of the Entity2's in accordance with such wish. Thus, it may be possible to have one, two, three, four or even more different labelling. Also, it may be possible to have one, two, three, four or even many more different probes targeting different or same regions.

When a multi-colour option is desired, the following

labelling compounds can suitably be used

fluorescein, and rhodamine,

fluorescein and Cy3,

fluorescein and lissamine,

fluorescein and coumarin,

rhodamine and coumarin,

lissamine and coumarin,

fluorescein, rhodamine, and coumarin,

fluorescein, lissamine, and coumarin.

In accordance herewith, dendrimers of the present invention

wherein both of the Entity1's and Entity2's are labelling compounds or probes substituted by labelling compounds,

wherein at least two, at least three, or at least four of the labelling compounds are different labelling compounds,

wherein the Entity1's are a labelling compound or a probe substituted by a labelling compound, and the Entity2's are another labelling compound or a probe substituted by said another labelling compound,

wherein both of the Entity1's and Entity2's are probes which may target different or same regions of the same or different targets,

wherein only the Entity1's are present,

wherein only the Entity2's are present,

as well as protected forms thereof,

are aspects of the present invention.

In the present context, the term “labelling compound” refers to a substituent which is useful for detection, i.e. suitable for generating a visible or otherwise detectable signal directly or indirectly. In accordance with the present invention, suitable labelling compounds comprise fluorophores, biotin, dinitro phenyl radicals, digoxigenin, radioisotope labels such as covalently bound radioisotope labels and complex bound radioactive ions, enzyme labels, dyes, chemiluminiscence labels, electroluminiscence labels, hapten, antigen or antibody labels, and spin labels. Examples of particular interesting labelling compounds are biotin, fluorescent labels, such as fluorescein labels, e.g. 5-(and 6)-carboxyfluorescein, 5- or 6-carboxyfluorescein, 6-(fluorescein)-5-(and 6)-carboxamido hexanoic acid and fluorescein isothiocyanate, dinitro phenyl radical, rhodamine, tetramethylrhodamine, cyanine dyes such as Cy2, Cy3 and Cy5, optionally substituted coumarin, R-phycoerythrin, allophycoerythrin, Texas Red and Princeston Red as well as conjugates of R-phycoerythrin and, e.g. Cy5 or Texas Red. Such labelling compounds may optionally be protected by one or more protecting groups. Examples of such protecting groups are given above.

In particular, the probe may be selected from peptide nucleic acids, RNA sequences or DNA sequences or analogues thereof, antibodies, antigens, proteins, peptides or derivatives thereof, epitopes, and biotin, or a protected form thereof.

In accordance with the definitions, the dendrimers of the present invention may thus have up to 2400 (x1 and x2 each being 1200) entities (Entity1's and Entity2's) attached thereto. The total number of entities (Entity1's and Entity2's) may suitably be up to 2000, 1000, 900, 800, 700, 600, 500, 400, 300, 200, 50, or 25. The number of Entity1's and Entity2's may be the same or different. In particular, the outermost layer of the dendrimer may have 4, 8, 16, 32, 64, 128, 256, 512, 1024, or 1048 entities attached thereto.

In a particular embodiment, the present invention relates to dendrimers having the general formula

(Entity1)ₓ₁-A-(Entity2)ₓ₂ (I)

wherein the dendritic core A is as defined above, and

wherein x1 and x2 independently is 2^{m}, m being an integer of from 1 to 10. In particular, m may be 2, 3, 4 or 5. Thus, m being 2 means that x1 and/or x2 is 4, m being 3 means that x1 and/or x2 is 8, m being 4 means that x1 and/or x2 is 16, or m being 5 means that x1 and/or x2 is 32.

In a special embodiment, the present invention relates to dendrimers, wherein the dendritic core A comprises one or more moieties of the structure (Ia)

Z[(Z]_{z}[)_{y}]_{z} (Ia)

wherein each Z is a group which contains at least one N-atomic branching point, said branching point not being part of a naturally occurring amino acid, and wherein the moiety of formula (Ia) comprises at least one ether group, and wherein each y independently is 2 or 3, and z is an integer of from 1 to 10, with the proviso that y^{z}≦1200, and protected forms thereof. Each Z group may be any group which provide spacing between the entities on the outermost layer of the dendrimer.

Within the scope of the present invention fall dendrimers, wherein Z groups contribute to the structure of the dendritic core A, wherein

x1 and x2 both a 0,

x1 is 0, and x2 is 1-1200,

x1 is 1-1200, and x2 is 0, and

x1 is 1-1200, and x2 is 1-1200,

as well as protected forms thereof.

An important embodiment is the dendrimers terminating in one or more groups (in principle x1 and/or x2 other groups) as described above.

In a special embodiment, the dendrimers of the present invention may be such, wherein the dendritic core A comprises at least two moieties of the structure (Ia) connected to a naturally or non-naturally amino acid, a peptide nucleic acid moiety, an LNA, a peptide, a protein, an antibody, an antigen, an immune complex, a DNA sequence or an analogue thereof, a RNA sequence or an analogue thereof, a macromolecule, and/or a solid or semi-solid support. A more detailed definition of these terms is given above. Accordingly, dendrimers are formed wherein each of the moieties (Ia) are connected to other compounds, thus forming the dendritic core A. These other compounds may suitably have connected thereto at least 2 moieties of the structure (Ia). However, in certain applications, many moieties of the structure (Ia) may be connected, such as from 2-100, 2-80, 2-70, 2-60, 2-50, 2-35, 2-30, 2-25, 2-20, 2-18, 2-15, 2-12, 2-10, 2-8,2-5, or 3 or 4. The moieties of the structure (Ia) may be connected terminally or internally to the other compounds as defined hereabove (e.g. alkyl groups, alkenyl groups, alkynyl groups, said groups containing substituents as defined above, amino acids, peptide nucleic acids, LNAs, peptides, proteins, and macromolecules).

In particular, each Z forming the moiety of the structure (Ia) and contributing to the dendritic core A may comprise one or more of C₁₋₁₀₀ alkyl groups, C₂₋₁₀₀ alkenyl groups, C₂₋₁₀₀ alkynyl groups, said alkyl, alkenyl, and alkynyl groups optionally containing one or more functional groups and/or one or more heteroatoms, naturally or non-naturally amino acids, peptide nucleic acid moieties, LNAs, peptides, proteins, antibodies, antigens, immune complexes, DNA sequences and analogues thereof, RNA sequences and analogues thereof, and macromolecules.

The alkyl, alkenyl and alkynyl groups may in particular be such with C₅₋₈₀, C₅₋₇₀, C₅₋₆₀, C₁₀₋₅₀, C₁₀₋₂₅, C₁₅₋₂₅, and C₁₀₋₂₀ carbon atoms which further optionally contains one or more functional groups and/or one or more heteroatoms. The terms “functional groups” and “heteroatoms” are defined above.

In particular, each Z forming the moiety of the structure (Ia) and contributing to the dendritic core A may comprise one or more of C₁₅₋₂₅ alkyl groups, C₁₅₋₂₅, alkenyl groups, of C₁₅₋₂₅ alkynyl groups, said alkyl, alkenyl, and alkynyl groups optionally one or more functional groups and/or one or more heteroatoms.

In a special embodiment, each Z contributing to the dendritic core A may comprise one or more of

—NH((CH₂)_{q}O))_{q}(CH₂)_{q}N((CH₂)_{q}C(O))₂,

(—CH₂)_{q}O)_{q}(CH₂)_{q}N((CH₂)_{q}O—)₂,

((—CH₂)_{q}O)_{q}(CH₂)_{q}C((CH₂)_{q}O—)₃, and

—NH((CH₂)_{q}O)_{q}(CH₂)_{q}C((CH₂)_{q}C(O)—)₃,

wherein each q independently is 0 an integer from 1 to 8, preferably from 1 to 3. Also comprised is successive layers containing one of more of these specified groups as well as one or more of naturally or non-naturally amino acids, peptide nucleic acid moieties, LNAs, peptides, proteins, antibodies, antigens, immune complexes, DNA sequences or analogues thereof, RNA sequences or analogues thereof, macromolecules, and solid or semi-solid supports, and further layers containing one or more of these specified groups and other layers containing one or more of naturally or non-naturally amino acids, peptide nucleic acid moieties, LNAs, peptides, proteins, antibodies, antigens, immune complexes, DNA sequences or analogues thereof, RNA sequences or analogues thereof, macromolecules, and solid or semi-solid supports.

In particular, each Z group forming the moiety of the structure (Ia) and contributing to the dentritic core A may comprise one or more of

—NH(CH₂)₃O(CH₂)₂O(CH₂)₂O(CH₂)₃N(CH₂C(O))₂—,

—NH(CH₂)₂NHC(O)CH₂(N(CH₂CH₂)N)CH₂C(O)—

(—NH(CH₂)₃(O)(CH₂)₂O(CH₂)₂O(CH₂)₃N(CH₂COOH)CH₂C(O))_{q}, wherein q is as defined above,

—NH(CH₂)₃O(CH₂)₂O(CH₂)₂O(CH₂)₃N(COOH)CH₂C(O)—

—NH(CH₃)₃(OCH₂CH₂)_{q}O(CH₂)₃N(CH₂COOH)CH₂C(O)—

—NH(CH₂)₃O(CH₂)₂O(CH₂)₂O(CH₂)₃NHC(O)CH₂O(CH₂CH₂)_{q}CH₂C(O)NH—(CH₂)₃O(CH₂)₂O(CH₂)₂O(CH₂)₃N(CH₂COOH)CH₂C(O)—.

In a particular embodiment, the dendritic core comprise one or more of the layer compounds ALL, DAAL, TAAL, PEG, PEG34, and PIP.

In particular, the Z groups contributing to the dendritic core A may be chosen so that that the interdistance between each of the Entity1 and/or each of the Entity2 or visa versa or visa versa is at least 5, at least 10, at least 20, at least 30, at least 35, at least 40, at least 50, at least 60, at least 70, at least 80, at least 90, at least 100, at least 110, at least 120, at least 130, at least 140, at least 150, at least 200, at least 210, at least 220, at least 230, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 525, at least 550, at least 575, or at least 600 bonds. In some embodiments, the interdistance is 35, 770, 130 and 250 bonds, cf. the Examples.

The dendrimers of the present invention, wherein Z groups contributes to the dendritic core A, can be constructed so as to present any combination of Entity1's and Entity2's. Particular interesting possibilities are

(1) the Entity1's are probes and the Entity2's are labelling compounds,

(2) the Entity1's are labelling compounds and the Entity2's are probes,

(3) both the Entity1's and the Entity2's are probes,

(4) both the Entity1's and the Entity2's are labelling compounds,

(5) the Entity1's are probes substituted by labelling compounds, and the Entity2's are labelling compounds,

(6) the Entity1's are labelling compounds, and the Entity2's are probes substituted by labelling compound,

(7) one or more of the Entity1's are probes, and the remaining Entity1's are another of the possible substituents as defined above, and the Entity2's are labelling compounds or probes substituted by labelling compounds,

(8) one or more of the Entity2's are probes, and the remaining Entity2's are another of the possible substituents as defined above, and the Entity1's are labelling compounds or probes substituted by labelling compounds,

(9) the Entity1's are the same or different labelling compounds or the same or different probes substituted by the same or different labelling compounds,

(10) the Entity2's are the same or different labelling compounds or the same or different probes substituted by the same or different labelling compounds,

(11) only the Entity1's are present (x1 being 0),

(12) only the Entity2's are present (x2 being 0), as well as protected forms thereof.

Of course one or more of the entities can be replaced by e.g. a reactive group, a free group, or an activated group, thereby terminating the dendritic core.

Thus, an aspect of the dendrimers wherein Z groups contributes to the dendritic core A include those

wherein one of the Entity1's and the Entity2's is a probe and the other is a labelling compound,

wherein both of the Entity1's and Entity2's are labelling compounds or probes substituted by labelling compounds,

wherein at least two, at least three, or at least four of the labelling compounds are different labelling compounds,

wherein the Entity1's are a labelling compound or a probe substituted by a labelling compound, and the Entity2's are another labelling compound or a probe substituted by said another labelling compound,

wherein both of the Entity1's and the Entity2's are probes,

wherein both of the Entity1's and Entity2's are probes which may target different or same regions of the same or different targets,

wherein only the Entity1's are present, and

wherein only the Entity2's are present.

The labelling compound is preferably selected from fluorophores, biotin, dinitro phenyl radical, digoxigenin, radioisotope labels, or enzyme labels, dyes, chemiluminiscence labels, hapten, antigen or antibody labels, and spin labels. Examples of such labelling compounds are given above. The labelling compound may be protected by a protecting group. Examples of suitable protecting groups are given above.

In particular, the probe may be selected from peptide nucleic acids, RNA sequences or DNA sequences or analogues thereof, antibodies, antigens, proteins, peptides or derivatives thereof, epitopes, and biotin.

Dendrimers of the present invention wherein Z groups contribute to the dendritic core A are suitably those wherein x1 and x2 independently is 2^{m}, and wherein m is an integer of from 1 to 10. In particular, m may be 2, 3, 4 or 5.

In general, in all the dendrimers of the present invention the Entity1's and Entity2's may be connected to terminally or internally to the substituents in question. Accordingly, at least one of the Entity1's and the Entity2's is connected to a naturally or non-naturally amino acid terminally or internally, a peptide nucleic acid terminally or internally, a peptide terminally or internally, an LNA terminally or internally, a protein terminally or internally, an antibody, an antigen, an immune complex, a RNA sequence or an analogues thereof terminally or internally, a DNA sequence or an analogue thereof terminally or internally, a macromolecule terminally or internally, or a solid or semi-solid support.

In a further aspect, the present invention relates to protected dendrimers as described herein.

The present invention also relates to dendrimer complexes comprising at least one dendrimer as defined herein connected to a naturally or non-naturally amino acid terminally or internally, a peptide nucleic acid terminally or internally, an LNA terminally or internally, a peptide terminally or internally, a protein terminally or internally, an antibody, an antigen, an immune complex, a RNA sequence or an analogue thereof terminally or internally, a DNA sequence or an analogue terminally or internally, a macromolecule terminally or internally, or a solid or semi-solid support,

and protected forms thereof.

In particular, the dendrimer complex is such, wherein the dendrimer is connected via at least one of the Entity1's and/or the Entity2's.

Another possibility is that in the dendrimer complex, the dendrimer is connected via a group or groups of the dendritic core A.

The dendrimer complexes of the invention may suitably have 1-100, 1-80, 1-70, 1-60, 1-50, 1-35, 1-30, 1-25, 1-20, 1-18, 1-15, 1-12, 1-10, 1-8,1-5, or 2, 3 or 4 dendrimers as defined herein connected thereto.

In a special embodiment, the dendrimer is internally connected to a naturally or non-naturally occurring amino acid, a peptide nucleic acid, an LNA molecule, a protein, a RNA sequence or a DNA sequence, a macromolecule, or a solid or a semi-solid support.

For example, when the dendrimer complex comprises a DNA or RNA sequence, a peptide nucleic acid, or a LNA molecule, one dendrimer as defined herein may be incorporated for each 50, 40, 35, 30, 25, 20, 15, 10, 5 amino acid.

In accordance with the above, non-limiting examples of the structures of interesting dendrimers/dendrimer complexes of the invention are

(1) (labelling compound)ₓ₁-dendritic core-(peptide nucleic acid, DNA, protein, and/or antibody connected to one or more alkyl, alkenyl, or alkynyl groups optionally containing functionalities/heteroatoms as defined above)-dendritic core-(labelling compound)ₓ₂.

In these types of compounds, Entity1's and Entity2's are labelling compounds, and the dendritic core A comprises as part thereof one ore more peptide nucleic acids, DNAs, proteins, and/or antibodies. The alkyl, alkenyl and alkynyl groups may be connected terminally or internally.

(2) (labelling compound)ₓ₁-dendritic core-(peptide nucleic acid, DNA, protein, and/or antibody)-dendritic core-(labelling compound)ₓ₂.

In these types of compounds, Entity1's and Entity2's are labelling compounds, and the dendritic core A comprises as part thereof one ore more peptide nucleic acids, DNAs, proteins, and/or antibodies.

(3) (labelling compound)ₓ₁-moiety of the structure (Ia)-(peptide nucleic acid, DNA, protein, and/or antibody)-moiety of the structure-(labelling compound)ₓ₂.

In these types of compounds, Entity1's and Entity2's are labelling compounds, and the dendritic core A comprises moieties of the structure (Ia) connected to one ore more peptide nucleic acids, DNAs, proteins, and/or antibodies.

(4) (labelling compound)ₓ₁-moiety of the structure (Ia)-(peptide nucleic acid, DNA, protein, and/or antibody connected to one or more alkyl, alkenyl, or alkynyl groups optionally containing functionalities/heteroatoms as defined above)-moiety of the structure-(labelling compound)ₓ₂.

In these types of compounds, Entity1's and Entity2's are labelling compounds, and the dendritic core A comprises moieties of the structure (Ia) connected to one ore more peptide nucleic acids, DNAs, proteins, and/or antibodies. The alkyl, alkenyl and alkynyl groups may be connected terminally or internally.

(5) (labelling compound)ₓ₁-dendritic core-(peptide nucleic acid, DNA, protein, and/or antibody connected to one or more alkyl groups, alkenyl groups, or alkenyl groups having substituents/functional groups as described above)-dendritic core-(labelling compound)ₓ₂.

In these types of compounds, Entity1's and Entity2's are labelling compounds, and the dendritic core A comprises as part thereof one ore more peptide nucleic acids, DNAs, proteins, and/or antibodies. The alkyl, alkenyl and alkynyl groups may be connected terminally or internally.

(6) (labelling compound)ₓ₁-moiety of the structure (Ia)-(peptide nucleic acid, DNA, protein, and/or antibody connected to one or more alkyl groups, alkenyl groups, or alkenyl groups having substituents/functional groups as described above)-moiety of the structure (Ia)-(labelling compound)ₓ₂.

In these types of compounds, Entity1's and Entity2's are labelling compounds, and the dendritic core A comprises moieties of the structure (Ia) connected to one or more peptide nucleic acids, DNAs, proteins, and/or antibodies.

(7) (protein or antibody)-dendritic core-(labelling compound)ₓ₂.

In these types of compounds, the Entity1's are a protein such as e.g. streptavidin or an antibody, and x1 is 1. The protein or antibody may be in the form of a monomer, dimer, trimer, tetramer or another multipler.

(8) (protein or antibody)-dendritic core-(labelling compound)ₓ₂.

In these types of compounds, the Entity1's are a protein such as e.g. streptavidin or an antibody, and x1 is 1. The dendritic core A comprises one or more of the moieties of the structure (Ia). The protein or antibody may be in the form of a monomer, dimer, trimer, tetramer or another multipler.

(9) (protein or antibody)-[dendritic core-(labelling compound)ₓ₂]_{1, 2, 3, . . .} .

In these types of compounds, the Entity1's are a protein such as e.g. streptavidin or an antibody, and x1 is 1. The protein or antibody may be in the form of a monomer, dimer, trimer, tetramer or another multiper. As indicated, these types of compounds may have one or more of the [dendritic core-(labelling compound)ₓ₂] attached thereto (dendrimer complex).

(10) (protein or antibody)-[dendritic core-(labelling compound)ₓ₂]_{1, 2, 3, . . .} .

In these types of compounds, the Entity1's are a protein such as e.g. streptavidin or an antibody, and x1 is 1. The protein or antibody may be in the form of a monomer, dimer, trimer, tetramer or another multiper. The dendritic core A comprises one or more of the moieties of the structure (Ia). As indicated, these types of compounds may have one or more of the [dendritic core-(labelling compound)ₓ₂] attached thereto (dendrimer complex).

(11) dendritic core-(labelling compound)ₓ₂.

In these types of compounds, x1 is 0. One or more of these dendrimers may be attached to a DNA, a peptide nucleic acid or an antibody via the dendritic core.

(12) dendritic core-(labelling compound)ₓ₂.

In these types of compounds, x1 is 0. The dendritic core A comprises one or more of the moieties of the structure (Ia). One or more of these dendrimers may be attached to a DNA, a peptide nucleic acid or an antibody via the dendritic core.

(13) (labelling compound attached to peptide nucleic acid, DNA, and/or protein)ₓ₁-dendritic core-(labelling compound)ₓ₂.

In these types of compounds, the labelling compounds may suitably be different, thus providing a multi-colour signal option.

(14) (labelling compound attached to peptide nucleic acid, DNA, and/or protein)ₓ₁-dendritic core-(labelling compound)ₓ₂.

The dendritic core A comprises one or more of the moieties of the structure (Ia). In these types of compounds, the labelling compounds may suitably be different, thus providing a multi-colour signal option.

(15) (labelling compound attached to peptide nucleic acid, DNA, and/or protein)ₓ-dendritic core-(peptide, protein, peptide nucleic acid, and/or DNA)ₓ₂.

In these types of compounds, the labelling compounds may suitably be different, thus providing a multi-colour signal option.

(16) (labelling compound attached to peptide nucleic acid, DNA, and/or protein)ₓ-dendritic core-(peptide, protein, peptide nucleic acid, and/or DNA)ₓ₂.

In these types of compounds, the labelling compounds may suitably be different, thus providing a multi-colour signal option. The dendritic core A comprises one or more of the moieties of the structure (Ia).

(17) The dendrimer complex may suitably comprise a protein such as tetrameric streptavidin or a dextran macromolecule having connected thereto several dendrimers as defined herein. The dendrimers may each suitably have attached thereto up to 4, 8, 16 or even 32 labelling compounds like fluorescein.

(18) The dendrimer complex may suitably comprise a protein such as tetrameric streptavidin or a dextran macromolecule having connected thereto several dendrimers as defined herein. The dendrimers may each suitably have attached thereto up to 4, 8, 16 or even 32 labelling compounds, wherein at least two of them are different, e.g. fluorescein and rhodamine.

The protected form of the above embodiments may be very interesting dendrimers and dendrimer complexes. Suitable protecting groups providing the protected dendrimer or dendrimer complex are those indicated above, in particular Boc, Fmoc, and Obz.

Although it is not specified in the all embodiments above, the labelling compounds may be the same or be different. Likewise, the probes may be the same or be different. Furthermore, the expression “a probe” is, as mentioned above, intended to include one or more of the possibilities mentioned under the definition of suitable probes, i.e. each Entity1 and/or Entity2 may comprise a combination of such probes. Likewise, the expression “a labelling compound” may comprise a combination of labelling compounds.

Protected dendrimer complexes as described herein also lie within the scope of the present invention.

As mentioned above, the application of the dendrimers and dendrimer/complexes are numerous.

Thus, in a further aspect, the present invention relates to the use of the dendrimers and dendrimer complexes as defined herein for detecting the presence of nucleic acid sequences, antibodies, antigens, immune complexes, proteins, or peptides in a sample.

The sample may be any sample to be tested, in particular a blood sample, a bone marrow sample, a chromosome spread, a tissue sample, a tissue section, a cell smear, a biopsy, an organ, a swap, a suspension of cells or parts thereof, and whole cells or parts thereof. The dendrimers are suitable for in vitro as well as in vivo diagnostic.

The dendrimers and dendrimer complexes of the invention are suitable for use in almost any assay system. Non-limiting examples are ELISA-based systems, dot blot, flow cytometry, in situ-based assays like ISH, other staining-based assays, other bead- or particle-based assays like turbidimetry, and slide-based systems.

A single dendrimer or dendrimer complex optionally displaying multi-functionalities may be used. Also several dendrimers or dendrimer complexes may be used. Or even a combination of dendrimers and dendrimer complexes may be used.

The dendrimers and dendrimer complexes of the present invention is as mentioned above suitable for the determination of substances present in biological samples. Non-limiting examples of application are given below.

1. For instance, Entity1 may be a probe capable of binding to specific nucleic acid sequences, immune complexes, antibodies, antigens or peptides in a sample, and Entity2 may be a labelling compound or a probe substituted by a labelling compound. This allows a strong enhancement of the signal.

2. Entity1 may be a probe capable of binding to specific nucleic acid sequences, immune complexes, antibodies, antigens or peptides in a sample, and one or more of the Entity2's may be a probe (e.g. DNA, peptide nucleic acid, or peptide) not binding to the substance in interest in the sample. Another dendrimer/dendrimer complex, wherein Entity1 is a probe binding specifically to Entity2, may then be applied. In this other dendrimer, Entity2 is suitably a labelling compound or a probe substituted by a labelling compound. A third or fourth dendrimer/dendrimer complex can of course be constructed in the same way. In this way, a multi-layer detection system is obtained. A strong enhancement of the signal is the result.

3. In particular when the dendrimer/dendrimer complex is used for detecting substances present in a low number of copies or even only a single copy, two or more dendrimers may be used. A dendrimer/dendrimer complex wherein Entity1 binds to a specific nucleic acid sequence in a sample, and a dendrimer wherein Entity1 is binding to another specific nucleic acid sequence in a sample in the vicinity of the first sequence, may be applied. The Entity2's may then be labelling compounds or probes substituted by labelling compound. Of course, additional dendrimers/dendrimer complexes may be constructed along the same pattern. This yields a strong amplification of the observed signal.

4. The principle of 3 may be extended to a multi-layer system as described above under 2.

5. The dendrimers/dendrimer complexes are particularly suitable for simultaneous detection of the sense and anti-sense strands of DNA. Entity1 may be chosen so as to bind to nucleic acid sequences of the sense strand, and Entity2 may be chosen so as to bind to nucleic acid sequences of the anti-sense strand. One or more of the Entity1 and/or Entity2 groups may be chosen so as to be labelling compound or probes substituted by labelling compounds making detection possible. Alternatively, a multi-layer system may be constructed in the same way as described above, i.e. one or more of Entity1's and/or Entity2's being probes not binding to the substance of relevance in the sample. This could e.g. be a different DNA, a peptide nucleic acid, a peptide, an antibody, or an antigen.

6. The dendrimer/dendrimer complexes may be conjugated to particles via Entity1's/Entity2's. One ore more Entity1's/Entity2's may then be a probe and the remaining labelling compounds.

7. The dendrimer/dendrimer complexes may contain labels in the dendritic core (which is possible if the dendritic core contains functional groups). Likewise, Entity1's/Entity2's may be labelled internally via functional groups in the probe.

8. Two or more Entity2's may be directed to sequences located nearby each other. Entity1's may then suitably be labelling compounds.

9. The dendrimer/dendrimer complex may suitably comprise one or more nucleic acid sequences and/or peptide nucleic acid moieties within the dendritic core.

10. The dendrimer complex may suitably comprise a protein such as tetrameric streptavidin or a dextran macromolecule having connected thereto several dendrimers as defined herein. The dendrimer complexes may each suitably have attached thereto up to 4, 8, 16 or even 32 labelling compounds like fluoresceine. Due to the loose structure of the dendrimers, no or little quenching occurs, and furthermore, the dendrimer/dendrimer complex still has good solubility. This of course yields a strong enhancement of the signal.

11. The dendrimer complex may suitably comprise a DNA strand having attached thereto a number of dendrimers. For instance, a dendrimer may be incorporated for each 10-15 base. If the dendrimers comprises labelling compounds, this provides a strong enhancement of the signal/amplification.

Another aspect of the present invention is detection systems comprising at least one dendrimer and/or at least one dendrimer complex as defined herein.

In a further aspect, the present invention relates to signal amplification systems comprising at least one dendrimer and/or at least one dendrimer complex as defined herein.

Accordingly, the present invention also relates to use of dendrimers and dendrimer complexes as defined herein as a detection system, as well as the use of dendrimers and a dendrimer complexes as defined herein as a signal amplification system.

Such detection systems or amplification systems are suitable for detecting the presence of nucleic acid sequences, antibodies, antigens, immune complexes, proteins, or peptides in a sample.

Furthermore, the dendrimers and dendrimer complexes of the present invention may be used in various labelling reactions. Thus, the present invention relates to the use of the dendrimers and dendrimer complexes as defined herein for labelling a compound. In particular, the compound to be labelled is selected from peptide nucleic acids, LNAs, RNA sequences, DNA sequences or analogues thereof, antibodies, antigens, immune complexes, proteins, peptides or derivatives thereof, epitopes, streptavidin, and biotin.

For the purpose of labelling reaction, the dendrimers and dendrimer complexes may suitably comprise one or more labelling compounds like fluorophores, biotin, dinitro phenyl radicals, digoxigenin, radioisotope labels, enzyme labels, dyes, chemiluminiscence labels, haptens, antigen or antibody labels, and spin labels.

In a further aspect, the present invention relates to labelling kits for labelling a compound, which kits comprise one or more dendrimers and/or dendrimer complexes. In an embodiment of such kit, the kit further comprises a labelling compound. In another embodiment, the kit is such, wherein the dendrimer or dendrimer complex comprises a labelling compound. The labelling compound may suitably be selected from fluorophores, biotin, dinitro phenyl radicals, digoxigenin, radioisotope labels, enzyme labels, dyes, chemiluminiscence labels, haptens, antigen or antibody labels, and spin labels.

It is also an object of the present invention to provide detection kits for detecting a compound, which kits comprise one or more dendrimers and/or one or more dendrimer complexes as defined herein, wherein the dendrimer and/or the dendrimer complex comprise(s) one or more labelling compounds. Of course, the dendrimers/dendrimer complexes can be unlabelled and in that case the kit may include means for labelling the dendrimers/dendrimer complexes. Suitable labelling compounds/means for labelling include fluorophores, biotin, dinitro phenyl radicals, digoxigenin, radioisotope labels, enzyme labels, dyes, chemiluminiscence labels, haptens, antigen or antibody labels, and spin labels.

The dendrimers of the present invention can be prepared by various methods, e.g. such used in connection with peptide synthesis. The person skilled in the art will readily know how to apply solid phase and liquid phase synthetic procedures when such are needed.

In particular, the building blocks of the dendrimer may be synthesised individually. Subsequently, the dendrimer may be assembled. This enables the preparation of the dendrimers as well as the protected forms.

Alternatively, the dendrimer may be synthesised by a step-by-step process, whereby the whole dendrimer is synthesised and the various entities at attached in a last step. This enables the preparation of the dendrimers as well as the protected forms.

In yet another process of preparation, the dendrimer can be synthesised step-wise in solution, whereby the product of the reaction is precipitated, and the procedure is repeated until the desired product is obtained.

For the synthesis, various solvents, reagents, protecting groups, activating agents etc. are to be applied. A synthetic strategy is outlined below and the reagents etc. mentioned in connection therewith can be applied in any other synthesis regime.

Thus, one process for preparing the dendrimers of the formula (I) or protected forms thereof as defined above comprises

preparing a dendritic core A by chemical/peptide synthesis, depending on the components of the dendritic core A, which dendritic core A has a number of protecting groups, which can be removed by deprotection,

if desired, removing the protecting groups by use of a suitable deprotecting agent,

if desired, coupling the Entity1's and/or the Entity2's to the dendritic core A, thereby obtaining the dendrimer.

Another process for preparing the dendrimers of the formula (I) or protected forms thereof comprises

preparing building blocks for the dendritic core A by chemical/peptide synthesis, depending on the components of the dendritic core A,

assembling the dendritic core A, which dendritic core A has a number of protecting groups, which can be removed by deprotection,

if desired, removing the protecting groups by use of a suitable deprotecting agent,

if desired, coupling the Entity1's and/or the Entity2's to the dendritic core A, thereby obtaining the dendrimer.

It is to be understood that the number of protecting groups may correspond to the number of Entity1's and Entity2's (i.e. the number of x1 and x2). However, fewer or more protecting groups are envisaged.

Yet another process for preparing the dendrimers of the invention or protected forms thereof comprises

coupling a starting material, which may be protected, to a substrate, e.g. a polyethylene glycol compound, which coupling product is soluble,

subjecting the coupling product to deprotection, if necessary, and a coupling reaction in solution,

precipitating the so formed product, and

if desired, repeating the above procedures,

thereby obtaining the dendrimer or the protected from thereof.

The protecting groups may be removed in one step or in more steps such as two, depending on whether orthogonal chemistries are used (see below). The reaction may optionally take place in a suitable solvent. The reaction temperature can be adjusted to facilitate the reaction.

The dendrimer complexes and the protected forms thereof of the present invention can be assembled by coupling procedures known to the person skilled in the art.

Thus, in a further aspect, the present invention relates to dendrimers and dendrimer complexes and protected forms thereof obtainable by the processes described herein.

Non-limiting examples of synthesis and assembly are given below.

One synthetic rational is described herebelow. Others are envisaged in the Examples.

A method for the preparation of a dendrimer having the formula (I)

(Entity1)ₓ₁-A-(Entity2)ₓ₂ (I)

wherein A is a dendritic core having at least one N-atomic branching point, said branching point not being part of a naturally occurring amino acid, and wherein A comprises at least one ether group,

each Entity1 and Entity2 are independently probes which optionally are substituted by one or more labelling compounds, a labelling compound, or a probe having reactive groups,

x1 is 1, and x2 is 0 or an integer of from 1 to 1200,

or a protected form thereof

which method comprises the steps of

(a) reacting a compound of the formula

Entity1-Z-(Entity2)_{y}

wherein Z is a group containing at least one branching point, Entity1 and Entity2 are suitable protecting groups which may be identical or different, and x2 is 2 or 3,

with a suitable deprotecting agent optionally in a suitable solvent, thereby obtaining a compound of the formula Entity1-Z and a compound of the formula Z-(Entity2)_{y},

wherein Entity1 and Entity2 are protecting groups,

(b) reacting the compounds obtained in step (a) with each other optionally in a suitable solvent, thereby obtaining a compound of the formula

Entity1-Z[Z-(Entity2)_{y}]_{y} (III)

wherein Entity1 and Entity2 are protecting groups,

(c) optionally deprotecting the compound obtained in step (b) with a suitable deprotecting agent optionally in a suitable solvent for further reaction, and optionally reacting the resulting compounds with each other or with one of the compounds of step (a) or (b),

(d) obtaining a resulting protected compound which may be deprotected for further reaction with a suitable deprotecting agent optionally in a suitable solvent,

(e) repeating steps (c) and (d) a desired number of times, thereby obtaining a compound of the formula

Entity1-A-(Entity2)ₓ₂ (IV)

wherein Entity1 and Entity2 are protecting groups, x2 is as defined above, and A is a dendritic core having the structure

Z[Z]_{z}[)_{y}]_{z} (Ia)

wherein z is as defined above,

(f) if desired, removing the protecting groups Entity1 and Entity2 simultaneously or individually with a suitable deprotecting agent optionally in a suitable solvent, and coupling the so obtained deprotected compound with Entity1 and Entity2, thereby obtaining the dendrimer of formula (I).

The dendrimers of the general formula (I)

(Entity1)ₓ₁-A-(Entity2)ₓ₂ (I)

wherein A is a dendritic core having at least one N-atomic branching point, said branching point not being part of a naturally occurring amino acid, and wherein A comprises at least one ether group,

each Entity1 and Entity2 are independently probes which optionally are substituted by one or more labelling compounds, a labelling compound, or a probe having reactive groups,

x1 and x2 are independently 0 or an integer of from 1 to 1200,

and protected forms thereof,

can be prepared by a method comprising the steps of

(f1) converting by deprotection one or more compounds of the general formula

Entity1-A-(Entity2)ₓ₂ (IV)

wherein A and x2 are as defined above, and Entity1 and Entity2 are suitable protecting groups,

with a suitable deprotecting agent optionally in a suitable solvent, into one or more compounds of the general formula

A-(Entity2)ₓ₂,

wherein Entity2 is a protecting groups,

(f2) reacting the compound of step (f1) with a compound of the formula A-(Entity1)ₓ₁, wherein Entity1 is a protecting group, or with each other, thereby obtaining a compound of the general formula

(Entity1)ₓ₁-A-(Entity2)ₓ₂ (V)

wherein x1 and x2 are as defined above, and Entity1 and Entity2 are protecting groups,

(f3) if desired, removing the protecting groups Entity1 and Entity2 simultaneously or individually with a suitable deprotecting agent optionally in a suitable solvent, and coupling the so obtained deprotected compound with Entity1 and Entity2 as defined above, thereby obtaining the dendrimer of formula (I).

In the above, the prefix “y” defines whether a given Z group is bifocated (y being 2) or trifocated (y being 3).

Within the present context, “protection groups” is intended to mean groups which can be removed with a suitable deprotection agent. Examples of suitable protection groups are Fmoc, Boc, Mtt, Dde, All, ODmab, Aloc, OtBu, OMe, OBz, Z, MOM, and benzyloxycarbonyl. The choice of protecting group depends i.a. on the type of group which need to be protected. Some of the above-indicated groups are suited for protecting —COOH groups, and others are suited for protecting —NH₂ groups. When two types of protecting groups are used, such are suitably selected so as to be orthogonal protecting groups. Furthermore, protecting groups should be selected so as to be compatible with the various entities on the intermediary compounds and/or the resulting dendrimer.

The term “suitable solvent” is intended to comprise solvents which allow the reaction to take place. Examples of suitable solvents are e.g. water, and organic solvents such as acetone, acetonitril, cyclohexane, DMSO, DMF, dichloromethane, NMP, DIPEA, benzene, and toluene, as well as mixtures thereof.

Within the present context, “suitable deprotecting agent” is intended to mean an agent allowing removal of the protecting groups. The choice of deprotecting agent depends on the choice of protecting groups. Examples of suitable deprotecting agents are TFA, TFMSA, HBr, HCl, HF, piperidine, DBU, hydrazine, base, nucleophiles, Pd/C/H₂, and Pd/AcOH/NMM.

The method of out lined above is described in greater detail below.

Step (a)

In step (a), a compound of the formula Entity1-Z-(Entity2)_{y}, wherein Entity1 and Entity2 are protecting groups, (a so-called dendrimer monomer) is reacted with a suitable deprotecting agent. The reaction may or may not take place in a solvent, i.e. the deprotecting agent may be sufficient to provide solubility. Suitable solvents are e.g. water, and organic solvents as defined above as well as mixtures thereof. If the Entity1-side of the Z group is an amine, suitable protecting groups are e.g. Boc, Fmoc, benzyloxycarbonyl, phathalimide, Mtt, Mmt, and trifluoroacetyl. If the Entity1 group is an acid, suitable protecting groups are e.g. benzyl, methyl, tert-butyl, MOM, and silyl esters. The same protecting groups are applicable for Entity2 having regard to whether an amine or an acid group is to be protected. The deprotecting agent(s) is/are selected so as to be compatible with the protection group to be removed. Removal of both deprotection groups can be accomplished at the same time e.g. by strong acid or HBr, or by mixing various deprotecting agents. The deprotection may be carried out at room temperature, under cooling e.g. on dry ice, or at an elevated temperature such as at from 100° C. to 180° C. Photochemical deprotection may also be an option.

In certain cases, it is advantageous to start out with two different reactions containers, each container containing the starting material, and in one removing the Entity1 protecting groups, and in the other removing the Entity2 protecting groups. Alternatively, the reaction can be carried out in the same reaction container, selecting the Entity1 and Entity2 protecting groups so as to not being removable by the same deprotecting agent. In either case, a compound of formula Entity1-Z and a compound of formula Z-(Entity2)_{y}, wherein Entity1 and Entity2 are protecting groups, are obtained.

Removal of the protecting groups imply that the Z group now has functional groups ready for further reaction, the number of functional groups depends on the possible number of branching points. Such functional groups include —COOH, —NH₂, cyclic anhydrides, thiols, alcohols, and halogens.

Two different starting compounds may be used, giving raise to dendrimers wherein the Z groups are mutually different.

Step (b)

In step (b), the compounds obtained in step (a) are reacted with each other, thereby yielding an intermediary compound of the formula (III) Entity1-Z[Z-(Entity2)_{y}]_{y}, wherein Entity1 and Entity2 are protecting groups. The reaction may be carried out at room temperature, under cooling e.g. on dry ice, or under heating to a temperature of from 100° C. to 180° C. Suitable solvents for the reaction include those mentioned above and mixtures thereof. However, the reaction need not be carried out in a solvent.

The product of this step is a protected dendrimer of formula (I). Upon removal of Entity2 protecting groups and conjugation of Entity2 groups, the dendrimer is formed. If y is 2, the dendrimer has four functionalities. If y is 3, the dendrimer has 2³=6 functionalities. Some of these may be in the free form, i.e. meaning that some of them do not carry a Entity2 group. If the protecting groups are not removed, a protected dendrimer is obtained.

As in step (a), removal of the protecting groups yields a compound having multiple functional groups which can be subjected to reaction.

The intermediary compound can, however, be subjected to further reaction extending the dendritic core. If this is desired, the process is proceeded according to step (c). Otherwise, the process proceeds according to step (f).

Step (c)

In step (c), the dendritic core is extended further. If further extension is not desired, the compound obtained in step (b) is subjected to reaction according to step (f). Thus, step (c) is an optional step.

The intermediary compound obtained in step (b) having the formula (III) Entity1-Z[Z-(Entity2)_{y}]_{y}, wherein Entity1 and Entity2 are protecting groups, is deprotected with a suitable deprotecting agent optionally in a suitable solvent. The deprotecting agent is selected so as to be capable of removing either the Entity1 protecting groups or the Entity 2 protecting groups, or, alternatively, both the Entity1 and the Entity2 protecting groups simultaneously. Accordingly, mixtures of deprotecting agents may be used. Suitable deprotecting agents are those given above.

Deprotection yields a compound having multiple functional groups for further reaction. Such functional groups are e.g. —COOH, —NH₂, cyclic anhydride, thiol, alcohols, and halogens.

The deprotection may optionally take place in a suitable solvent. Examples of suitable solvents or mixtures of solvents are as described above (step (a) and (b)). The choice of solvent/solvents depends on the choice of protecting groups and reaction conditions.

The reaction may take place at room temperature, under cooling e.g. on dry ice, or with heating. Photochemical reaction is also possible.

The deprotection can be carried out in two reaction containers as described above., one in which Entity1 is removed, yielding a compound of the formula Z[Z-(Entity2)_{y}]_{y}, and one in which Entity2 is removed, yielding a compound of the formula Entity1-Z[Z]Y, wherein Entity1 and Entity2 are protecting groups, and wherein each [Z] has y functional groups available for further reaction. As apparent from the formula, there are y such [Z] groups.

The compounds so obtained can be reacted with each other, or can be reacted with one of the compounds of step (a) or (b). Alternatively, the deprotected compound can be reacted with a compound having one or more mutually different Z groups, thus giving raise to dendrimers in which the various Z groups are mutually different.

If the deprotected compound is reacted with a compound in which the Entity1 protecting group is removed, the reaction gives raise to a dendrimers of formula (I) having a plurality of Entity1's and Entity2's upon conjugation with such entities. If the protecting groups are not removed, a protected dendrimer is obtained.

If the deprotected compound is reacted with a compound in which the Entity2 protecting group is removed, the reaction gives raise to dendrimers of formula (I) upon conjugation with Entity1 and Entity2.

Step (d)

In step (d), the protected compound obtained in step (c) may be deprotected for further reaction. Deprotection optionally takes place in a suitable solvent. Examples of suitable solvents are those mentioned above under step (a), (b) and (c). Deprotection is carried out using suitable deprotecting agents selected so as to be compatible with the protecting groups to be removed, further having regard to whether different protecting groups are to be removed. Examples of suitable deprotecting agents are given above.

Step (e)

In step (e), deprotecting and reaction are repeated a desired number of times until the dendritic core has the desired size. Thus, an intermediary compound of formula (IV) Entity1-A-(Entity2)ₓ₂, wherein Entity1 and Entity2 are protecting groups, is obtained.

When one or more dendrimers of the desired form is/are obtained, such can be attached to one or more nucleic acid or DNA sequences and/or peptide nucleic acid moieties, thereby obtaining a dendrimer having such sequences within the dendritic core.

Step (f)

In step (f), removal of the Entity1 and Entity2 protecting groups is accomplished. Removal of the Entity1 and Entity2 protecting groups can be carried out at the same time, or in different steps. This depends i.a. on the choice of deprotecting agent and the choice of protecting groups. Suitable deprotecting agents are indicated above. The reaction may or may not take place in a suitable solvent or mixtures of such solvents. Examples of suitable solvents are given above.

Subsequently, the deprotected intermediary compound is coupled to suitable Entity1's and Entity2's resulting in a dendrimer of formula (I). Coupling conditions are selected so as to be compatible with the nature of Entity1's and Entity2's. Coupling may suitably be carried out using a suitable coupling agent such as carbodiimides, HBTU, HATU, TBTU, BOP, PyBOP, PyAOP, DhbtOH esters, Pfp, Bt, and At. Coupling may optionally take place in a solvent or a mixture of various solvents such as those mentioned above. Coupling may be carried out under room temperature, under cooling, under heating, or by means of a photochemical reaction. Coupling may be accomplished by converting the dendrimer to the activated form that is very reactive, e.g. an anhydride, a haloacetyl derivative and other halogenides, thiols, maleimides, and NHS esters.

Step (f1)

In step (f1), an intermediary compound of the formula (IV) Entity1-A-(Entity2)ₓ₂, wherein Entity1 and Entity2 are protecting groups, is deprotected using a suitable deprotecting agent. Suitable deprotecting agents are e.g. those given above. The reaction may or may not take place in a suitable solvent or a mixture of such solvents. The reaction may be performed at room temperature, under cooling e.g. on dry ice, or with heating to about 100-180° C.

The purpose is to obtain a dendrimer of the formula (I) which has multiple entity presenting capabilities at both “sides” of the dendritic core.

By removal of the Entity1 protecting groups, a compound of the formula A-(Entity2)ₓ₂, wherein Entity2 is a protecting group, is obtained.

If a dendritic core of a smaller size is desired, the intermediary compound of formula (III) obtained in step (b) can be subjected to reaction (step (c)) with a compound wherein the Entity1 protecting groups has also been removed. In this way, a protected dendrimer of formula (I) is also obtained, however, having a smaller dendritic core compared to the dendrimer of formula (II) obtained by reaction of the intermediary compound of formula (IV).

Step (f2)

In step (f2), the deprotected compound of step (f1) is subjected to reaction with another intermediary compound of formula (IV) in which Prot1 has been removed. Alternatively, the deprotected compound of step (f1) can be subjected to reaction with another compound in which Prot1 has been removed leading to dendrimers in which the Z groups are mutually different.

As for the other above-indicated steps, the reaction can take place with or without solvent. The reaction can be performed at room temperature, under cooling or heating, or by photochemical reaction.

Suitable deprotecting agents are e.g. those given above.

By this reaction, an intermediary compound of the formula (V), (Entity1)ₓ₁-A-(Entity2)ₓ₂, wherein Entity1 and Entity2 are protecting groups, is obtained.

Step (f3)

In step (f3), the protecting groups are removed. By removal of the protecting groups, functional groups which are reactive are formed (activated groups). Such functional groups are e.g. —COOH, —NH₂, cyclic anhydride, thiols, alcohols, and halogens. Removal of the Entity1 and Entity2 protecting groups can be performed at the same time or in multiple steps.

Subsequently, coupling with Entity1 and Entity2 are performed. Coupling is performed as described above under step (f).

The Entity1 and Entity2 protecting groups may be mutually different or identical. For most purposes, it is most suitable that the Entity1 and Entity2 protecting groups are mutually different since this allows coupling of Entity1's and Entity2's which are mutually different.

In all the above-described steps, it may be necessary to protect sensitive side-chain groups. This can be done using suitable temporary side-chain protecting groups such as Boc, tBu, Z, and Cbz. Side-chain protective groups may be removed by means of an acid such as TFA, TFMSA, HBr, HCl or HF, a base, or a nucleophil like pyridine.

Compared with conventional procedures for obtaining dendritic compounds, the present method is a liquid phase reaction, whereas most conventional procedures are based on solid-phase techniques.

Within the scope of the present invention is also the various intermediary compounds. Furthermore, the present invention also encompasses dendrimers and intermediary obtainable by such manufacturing methods.

The invention is further illustrated by the following, non-limiting examples.

### EXAMPLES

### Example 1

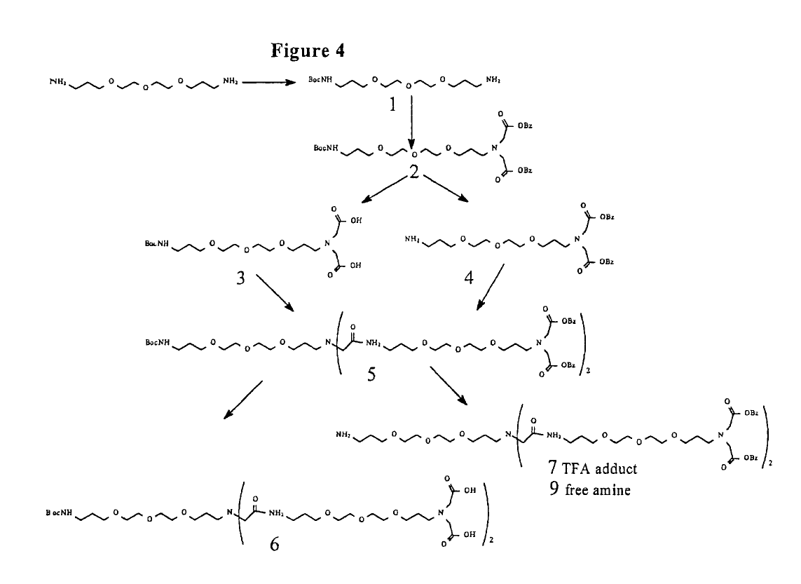

Preparation of 1.1-(tert-butoxycarbonyl)-1,15-diaza-4,7,10-trioxapentadecane; cf. FIG. 4, No. 1

21.8 g, 0.1 mol, (Boc)20 dissolved in dioxane (200 ml) was added drop-wise to 110 g, 0.5 mol, 1,13-diamino-4,7,10-trioxatridecane dissolved in dioxane (400 ml). Following 20 minutes stirring after addition and 1 of h additional stirring, the solvent was evaporated, and the resulting oil partitioned between water (300 ml) and dichlorometane (300 ml). The organic phase was reduced to an oil and purified by column chromatography on silica. The product eluted (r.f. 0.5) with 10% ammonia saturated methanol in dichloromethane. Yield 23.0 g colourless oil (72%). Mw. calculated for C₁₅H₃₂N₂O₅ 320.3. Found 321.9 (MH⁺, MALDI-TOF MS).

### Example 2

Preparation of 1-(tert-butoxycarbonyl)-1,15-diaza-4,7,10-trioxapentadecane-15-bis(benzyloxycarbonylmethyl), FIG. 4, No. 2

Protected dendrimer monomer having the form Entity1-A-(Entity2)₂, dendritic core A=—NH(CH₂)₃O(CH₂)₂O(CH₂)₂O(CH₂)₃₋N(CH₂C(O))₂—, Entity1=Boc, Entity2=OBz, x1=1, x2=2

6.4 g, 0.02 mol, 1-(tert-butoxycarbonyl)-1,15-diaza-4,7,10-trioxapentadecane was dissolved in DMF (25 ml) and DIPEA (13 ml). 2-bromoaceticacid benzyl ester, 10.0 g, 4.3 mol, was added, and after 5 minutes the reaction mixture was further heated to 80° C. for 35 min. The solvents were evaporated, the residue partitioned between saturated NaHCO₃ (500 ml) and ethyl acetate (500 ml). The organic phase was washed with water (2×500 ml), evaporated to dryness and purified on silica, and the product was eluted (r.f 0.5 with 1:1 ethylacetate/hexane). Yield 9.9 g pale yellow oil (80%). Mw calculated for C₃₃H₄₈N₂O₉ 616.8. Found 618.2 (MH⁺, MALDI-TOF MS).

### Example 3

Preparation of 3.1-(tert-butoxycarbonyl)-1,15-diaza-4,7,10-trioxapentadecane-15-diacetic Acid, FIG. 4, No. 3

Deprotected dendrimer monomer having the form Entity1-A-(free acid)₂, A=—NH(CH₂)₃O(CH₂)₂O(CH₂)₂O(CH₂)₃N(CH₂C(O))₂—, Entity1=Boc, x1=1, dendritic core terminates in a free acid

3.09 g, 5 mmol, 1-(tert-butoxycarbonyl)-1,15-diaza-4,7,10-trioxapentadecane-15-bis(benzyloxycarbonylmethyl) was dissolved in methanol (30 ml). 10% Pd/C (300 mg) was added, and the reaction mixture hydrogenated (deprotection) at room temperature and room pressure for 1 h. Pd/C was spun down, and the supernatant was reduced to dryness. Yield 2.2 g colourless resin (99%). Mw. calculated for C₁₉H₃₆N₂O₉ 436.5. Found 437.9 (MH⁻, MALDI-TOF MS).

### Example 4

Preparation of 4.1,15-diaza-4,7,10-trioxapentadecane-15-bis(benzyloxycarbonylmethyl) (TFA Adduct), FIG. 4, No. 4

Dendrimer having formula (free amine)-A-(Entity2)₂, A=—NH(CH₂)₃O(CH₂)₂O(CH₂)₂O(CH₂)₃N(CH₂C(O))₂—, Entity2=OBz, x2=2, dendritic core terminates in free amine form

6.78 g, 11 mmol, 1-(tert-butoxycarbonyl)-1,15-diaza-4,7,10-trioxapentadecane-15-bis(benzyloxycarbonylmethyl) was evaporated twice to dryness from TFA (2×20 ml). Yield approximately 13 g of TFA adduct as a pale yellow oil (100%). Mw. calculated for C₂H₄₀N₂O₇ 516.5. Found 517.9 (MH⁺, MALDI-TOF MS).

### Example 5

Preparation of 1-(tert-butoxycarbonyl)-1,15-diaza-4,7,10-trioxapentadecane-15-bis[3,17-diaza-2-oxo-7,10,13-trioxaheptadecane-17-bis (benzyloxycarbonylmethyl)], FIG. 4, No. 5

Protected dendrimer having the form Entity1-A-(Entity2)₂, dendritic core A=three —NH(CH₂)₃O(CH₂)₂O(CH₂)₂O(CH₂)₃ N(CH₂(C(O))₂— groups, Entity1=Boc, Entity2=OBz, x1=1, x2=4

13 g, 11 mmol, 1,15-diaza-4,7,10-trioxapentadecane-15-bis-(benzyloxycarbonylmethyl) (TFA adduct) was dissolved in DMF (25 ml) and triethylamine (7.5 g). 2.2 g, 4.9 mmol, 1-(tert-butoxycarbonyl)-1,15-diaza-4,7,10-trioxapentadecane-15-diacetic acid in DMF (4 ml) was added, followed by 6.24 g, 12 mmol, PyBOP. The mixture was stirred for 2 h at 45° C., the solvents were evaporated. The residue was partitioned between saturated NaHCO₃ (200 ml) and DCM (500 ml). The DCM phase was further extracted twice with water (2×200 ml) and reduced to dryness. The resulting oil was purified on silica, and eluted (r.f. 0.5) with 15% methanol in ethyl acetate. Yield 3.7 g as a viscous oil (53%). Mw. calculated for C₇₅H₁₁₂N₆O₂₁ 1433.8. Found 1435.1 (MH⁺, MALDI-TOF MS).

### Example 6

Preparation of 1-(tert-butoxycarbonyl)-1,15-diaza-4,7,10-trioxapentadecane-15-bis[3,17-diaza-2-oxo-7,10,13-trioxaheptadecane-17-diacetic Acid], FIG. 4, No. 6

Partly protected dendrimer of the structure Entity1-A-(free acid)₄ (IV), dendritic core A comprising 3 groups —NH(CH₂)₃O(CH₂)₂O(CH₂)₂O(CH₂)₃N(CH₂C(O))₂—, Entity1=Boc, x1=1, dendritic core terminates in free acid form —COOH

1.43 g, 1 mmol, 1-(tert-butoxycarbonyl)-1,15-diaza-4,7,10-trioxapentadecane-15-bis[3,17-diaza-2-oxo-7,10,13-trioxaheptadecane-17-bis (benzyloxycarbonylmethyl)] was dissolved in methanol (15 ml). 10% Pd/C (150 mg) was added, and the reaction mixture hydrogenated at room temperature and room pressure for 1 h. Pd/C was spun down, and the supernatant reduced to dryness. Yield 1.04 g as a viscous oil (97%). Mw. calculated for C₄₇H₈₈N₆O₂₁ 1073.2. Found 1075.2 (MH⁺, MALDI-TOF MS).

### Example 7

Preparation of 1.15-diaza-4,7,10-trioxapentadecane-15-bis[3,17-diaza-2-oxo-7,10,13-trioxaheptadecane-17-bis-benzyloxycarbonylmethyl)], FIG. 4, No. 7

Partly protected dendrimer of formula (free amine)-A-(Entity2)₂, dendritic core A comprising 3 moieties of formula —NH(CH₂)₃O(CH₂)₂O(CH₂)₂O(CH₂)₃N(CH₂C(O))₂—, Entity2=OBz, x2=4, dendrimer terminates in free amine form —NH₂

7.15 g, 5 mmol, 1-(tert-butoxycarbonyl)-1,15-diaza-4,7,10-Trioxapentadecane-15-bis[3,17-diaza-2-oxo-7,10,13-trioxaheptadecane-17-bis(benzyloxycarbonylmethyl)] was evaporated from TFA twice to dryness (2×30 ml). Yield approximately 15 g of TFA adduct as a pale yellow oil (100%). Mw. calculated for C₇₀H₁₀₄N₆O₁₉ 1333.6. Found 1334.1 (MH⁺, MALDI-TOF MS).

### Example 8

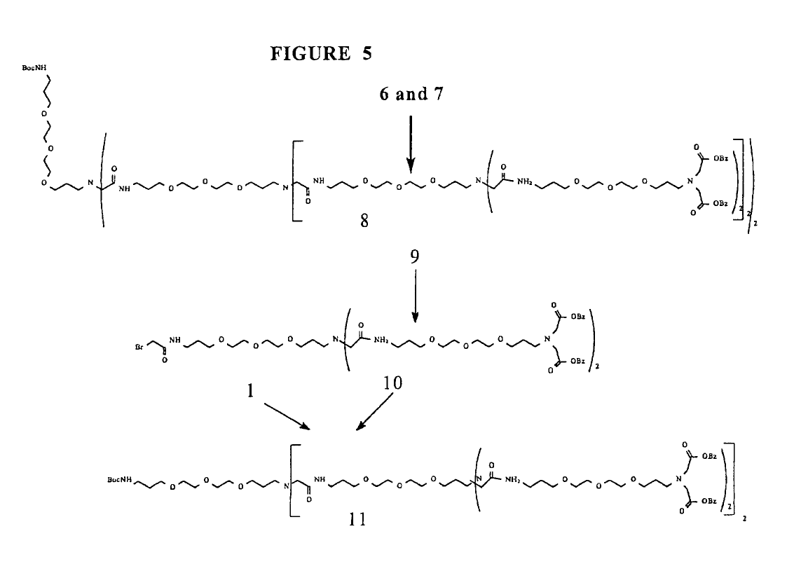

Preparation of 1-(tert-butoxycarbonyl)-1,15-diaza-4,7,10-trioxapentadecane-15-bis[3,17-diaza-2-oxo-7,10,13-trioxaheptadecane-17-bis(3,17-diaza-2-oxo-7,10,13-trioxaheptadecane-17-bis[3,17-diaza-2-oxo-7,10,13-trioxaheptadecane-17-bis(benzyloxycarbonylmethyl)])], FIG. 5, No. 8

Protected dendrimer of formula Entity1-A-(Entity2)₈, dendritic core A comprising 15 moieties of formula —NH(CH₂)₃O(CH₂)₂O(CH₂)₂O(CH₂)₃N(CH₂C(O))₂—, Entity1 Boc, Entity2=OBz, x1=1, x2=16

15 g, 5 mmol, 1,15-diaza-4,7,10-trioxapentadecane-15-bis-[3,17-diaza-2-oxo-7,10,13-trioxaheptadecane-17-bis(benzyloxycarbonylmethyl)] (TFA adduct) was dissolved in DMF (30 ml) and triethylamine (9 g). To this solution was added 1.04 g, 0.97 mmol, 1-(tert-butoxycarbonyl)-1,15-diaza-4,7,10-trioxapentadecane-15-bis[3,17-diaza-2-oxo-7,10,13-trioxaheptadecane-17-diacetic acid] followed by 3.12 g, 6 mmol, PyBOP. The mixture was stirred at 45° C. for 2 h and the solvents were evaporated. The residue was taken up in DCM (200 ml) and methanol (20 ml) and extracted with saturated NaHCO₃ (200 ml), followed by water (2×50 ml). The organic phase was evaporated to dryness and purified on silica, eluding (r.f. 0.3) with 15% methanol in DCM. Yield 2.1 g colourless oil (33%). Mw. calculated for C₃₂₇H₄₉₆N₃₀O₉₃ 6335.7. Found 6333.6 (MH⁺, MALDI-TOF MS)

### Example 9

Preparation of 1,15-diaza-4,7,10-trioxapentadecane-15-bis [3, 17-diaza-2-oxo-7, 10, 13-trioxaheptadecane-17-bis-(benzyloxycarbonylmethyl)], FIG. 5, No. 9

Partly protected dendrimer of formula (free amine)-A-(Entity2)₂, dendritic core A comprising 3 moieties of formula —NH(CH₂)₃O(CH₂)₂O(CH₂)₂O(CH₂)₃N(CH₂C(O))₂—, Entity2=OBz, x2=4, dendrimer terminates in free amine form —NH₂

1,15-diaza-4,7,10-trioxapentadecane-15-bis[3,17-diaza-2-oxo-7,10,13-trioxaheptadecane-17-bisbenzyloxycarbonylmethyl)] 15 g, (5 mmol of TFA adduct, cf. Example 7) was dissolved in DCM (150 ml) and neutralised with 0.5 M Na₂CO₃ (150 ml). The organic phase was isolated. Toluene (50 ml) was added, and the solvents were evaporated. Yield 5.7 g as a viscous oil (85%). Mw. calculated for C₇₀H₁₀₄N₆O₁₉ 1333.6. Found 1334.9 (MH+, MALDI-TOF MS). This compound was observed to undergo slow ammonolysis at room temperature.

### Example 10

Preparation of 1-(2-bromoacetyl)-1,15-diaza-4,7,10-trioxapentadecane-15-bis[3,17-diaza-2-oxo-7,10,13-trioxaheptadecane-17-bis(benzyloxycarbonylmethyl)], FIG. 5, No. 10

Activated dendrimer of the form (activated group)-A-(Entity2)₄, activated group=bromoacetyl, Entity2=OBz, x2=4, dendritic core terminates in an activated group

1,15-diaza-4,7,10-trioxapentadecane-15-bis[3,17-diaza-2-oxo-7,10,13-trioxaheptadecane-17-bis-(benzyloxycarbonylmethyl)], 9, 5.7 g, 4.25 mmol, in toluene (25 ml) was added drop-wise over 20 minutes to bromoacetic anhydride 1.3 g, 5 mmol, and lutidine 870 mg, 8 mmol, in ice bath cooled DCM (25 ml). Further DCM (100 ml) was added to the mixture, which was then extracted with NaHCO3 (100 ml). The organic phase was filtered and evaporated to dryness. The resulting oil was purified on silica, eluding (r.f. 0.5) with 10% methanol in DCM. Yield 5.25 g as a viscous oil (85%). Mw. calculated for C₇₂BrH₁₀₅N₆O₂₀ 1454.7. Found 1456.1 (MH+, MALDI-TOF MS).

### Example 11

Preparation of 1-(tert-butoxycarbonyl)-1,15-diaza-4,7,10-trioxapentadecane-15-bis(3,17-diaza-2-oxo-7,10,13-trioxaheptadecane-17-bis-[3,17-diaza-2-oxo-7,10,13-trioxaheptadecane-17-bis-(benzyloxycarbonylmethyl)]) FIG. 5, No. 11

Protected dendimer of the form Entity1-A-(Entity2)₈, Entity1=Boc, Entity2=OBz, x1=1, x2=8, dendritic core A comprising 7NH(CH₂)₃O(CH₂)₂(CH₂)₂O(CH₂)₃N(CH₂C(O))₂—

1-(2-bromoacetyl)-1,15-diaza-4,7,10-trioxapentadecane-15-bis[3,17-diaza-2-oxo-7,10,13-trioxaheptadecane-17-bis-(benzyloxycarbonylmethyl)], 5.25 g, 3.6 mmol, and 1-(tert-butoxycarbonyl)-1,15-diaza-4,7,10-trioxapentadecane, 580 mg 1.8 mmol was dissolved in DMF (6 ml) and DIPEA (1 ml). The mixture was stirred at 80° C. for 2 hours, and the solvents were removed. The resulting oil was taken up in DCM (100 ml) which was extracted with saturated NaHCO₃ (100 ml) then washed with water (2×20 ml). After solvent removal the product was purified on silica, eluding (r.f. 0.3) with 10% methanol in DCM. Yield 4.5 g as a viscous oil (81%). Mw calculated for C₁₅₉H₂₄₀N₁₄O₄₅ 3067.73. Found 3069.2 (MH+, MALDI-TOF).

### Example 12

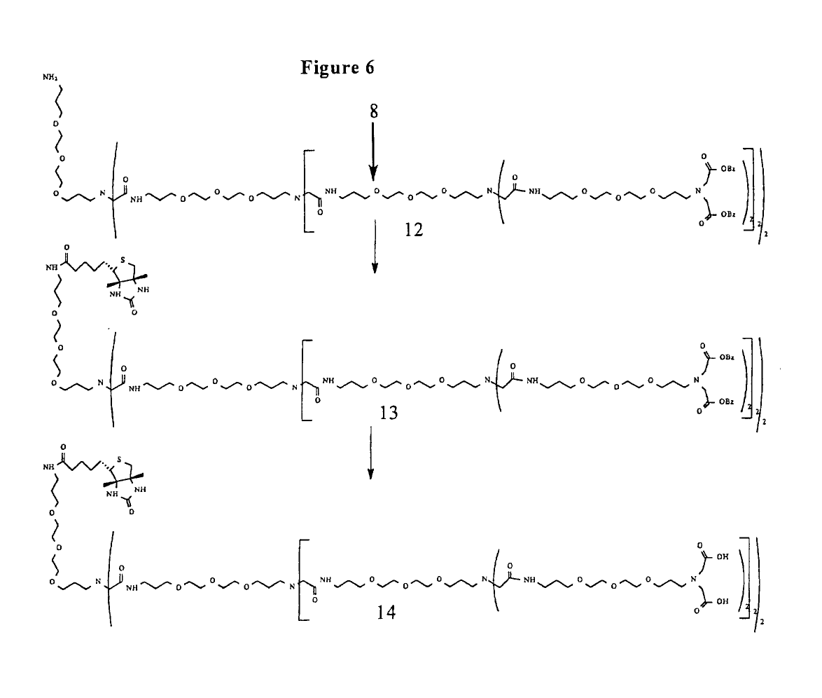

Preparation of 1-(tert-butoxycarbonyl)-1,15-diaza-4,7,10-trioxapentadecane-15-bis[3,17-diaza-2-oxo-7,10,13-trioxaheptadecane-17-bis(3,17-diaza-2-oxo-7,10,13-trioxaheptadecane-17-bis[3,17-diaza-2-oxo-7,10,13-trioxaheptadecane-17-bis(benzyloxycarbonylmethyl)])], FIG. 6, No. 12

Partly protected dendrimer of the form (free amine)-A-(Entity2)₁₆, dendritic core A comprising 15 moieties of formula —NH(CH₂)₃O(CH₂)₂O(CH₂)₂O(CH₂)₃N(CH₂C(O))₂—, Entity2=OBz, x2=16, x1=0, dendritic core terminates in a free amine

The compound of Example 8 (100 mg) was dissolved in TFA (1 ml) and after 5 minutes, the product could be isolated by precipitation with ether. The yield was assumed to be quantitative. Mw. calculated for C₃₂₂H₄₈₈N₃₀O₉₁ 6235.7. Found 6237.3 (MH⁺, MALDI-TOF MS)

### Example 13

Preparation of 1-(biotinyl)-1,15-diaza-4,7,10-trioxapentadecane-15-bis[3,17-diaza-2-oxo-7,10,13-trioxaheptadecane-17-bis(3,17-diaza-2-oxo-7,10,13-trioxaheptadecane-17-bis [3,17-diaza-2-oxo-7,10,13-trioxaheptadecane-17-bis-(benzyloxycarbonylmethyl)])], FIG. 6, No. 13

Derivatised dendrimer of the form Entity1-A-(Entity2)₁₆, dendritic core A comprising 15 moieties of formula —NH(CH₂)₃O(CH₂)₂(CH₂)₂O(CH₂)₃N(CH₂C(O))₂—, Entity1=biotinyl, Entity2=OBz, x1=1, x2=16

The compound of Example 12 (100 mg) was dissolved in NMP (1 ml) and DIPEA (0.1 ml), and biotin (5 mg) was added followed by PyBOP (8 mg). After 1 hour at 45° C., the crude product was isolated by precipitation with ether (8 ml). The so obtained compound was re-dissolved in 10 ml DCM and extracted with NaHCO₃ (10 ml). The organic phase was reduced to dryness to yield the product. Yield 80 mg (79%). Mw. calculated for C₃₃₂H₅₀₂N₃₂O₉₃S 6461.8. Found 6463.2 (MH⁺, MALDI-TOF MS).

### Example 14

Preparation of 1-(biotinyl)-1,15-diaza-4,7,10-trioxapentadecane-15-bis[3,17-diaza-2-oxo-7,10,13-trioxaheptadecane-17-bis(3,17-diaza-2-oxo-7,10,13-trioxaheptadecane-17-bis[3,17-diaza-2-oxo-7,10,13-trioxaheptadecane-17-bis-(carboxymethyl)])], FIG. 6, No. 14

Dendrimer of the form Entity1-A-(free acetic acid)₁₆, dendritic core A comprising 15 moieties of formula —NH(CH₂)₃O(CH₂)₂O(CH₂)₂O(CH₂)₃N(CH₂C(O))₂—, Entity1=biotinyl, x1=1, dendrimer terminates in free acetic acids

The compound of Example 13 (80 mg) was dissolved in 2 ml methanol and 30 mg Pd/C was added. The mixture was hydrogenated under atmospheric pressure for 1 hour. The catalyst was filtered off, and the mixture reduced to dryness. Yield: 62 mg (100%). Mw. calculated for C₂₂₀H₄₀₆N₃₂O₉₃S 5019.7. Found 5022.3 (MH⁺, MALDI-TOF MS).

### Example 15

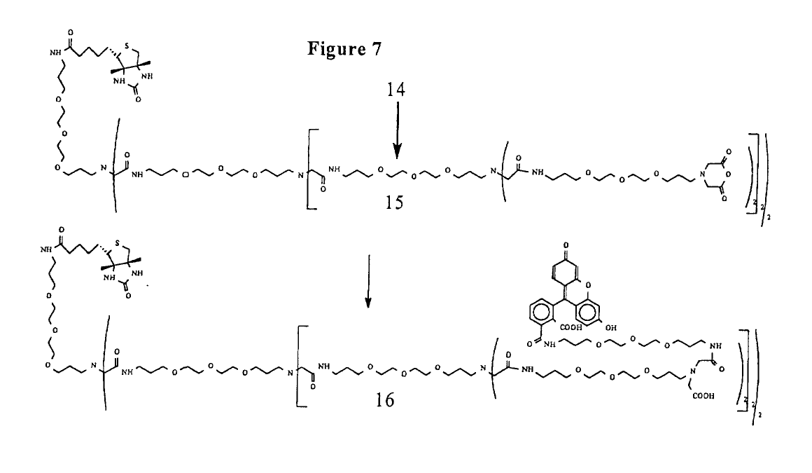

Preparation of 1-(biotinyl)-1,15-diaza-4,7,10-trioxapentadecane-15-bis[3,17-diaza-2-oxo-7,10,13-trioxaheptadecane-17-bis(3,17-diaza-2-oxo-7,10,13-trioxaheptadecane-17-bis[3,17-diaza-2-oxo-7,10,13-trioxahexadecane-16-(2,6-dioxomorpholin-4-yl)])], FIG. 7, No. 15

Activated dendrimer of the form Entity1-A-(activated group)₈, dendritic core A comprising 17 moieties with 7 —NH(CH₂)₃O(CH₂)₂O(CH₂)₂O(CH₂)₃N(CH₂C(O))₂—, and the peripheral 8 groups —NH(CH₂)₃O(CH₂)₂O(CH₂)₂O(CH₂)₃-(2,6-dioxomorpholin-4-yl)-, Entity1=biotinyl, x1=1, dendrimer terminates in N-2,6-dioxymorpholine

The compound of Example 14 (10 mg) was dissolved in pyridine (100 μl) and diisopropylcarbodiimide (4 μl) was added. After 30 minutes at 55° C., the product was precipitated by addition of ether. Yield: 9 mg (91%). Mw. calculated for C₂₂₀H₃₉₀N₃₂O₈₅S 4875.7. Found 4876.5 (MH⁺, MALDI-TOF MS).

### Example 16

Final Coupling of Dendrimer from Example 15, FIG. 7, No. 16

Dendrimer of formula of the form Entity1-A-(Entity2)₈, dendritic core A comprising 15 groups with 7 —NH(CH₂)₃O—(CH₂)₂O(CH₂)₂O(CH₂)₃N(CH₂C(O))₂—, and the peripheral 8 groups=—NH(CH₂)₃O(CH₂)₂O(CH₂)₂O(CH₂)₃—(N(CH₂COOH)(CH₂C(O)—)), Entity1=biotinyl, Entity2=linker-carboxyfluorescein wherein linker=—NH(CH₂)₃O(CH₂)₂O(CH₂)₂O(CH₂)₃NH—, x1=1, x2=8

The compound of Example 15 (9 mg) was dissolved in NMP (100 μl), and DIPEA (20 μl) and (13-carboxyfluoresceinylamido)-4,7,10-trioxotridecane-1-amine (13 mg) were added. After 10 minutes at 55° C., the crude product was isolated by precipitation with ether. Yield: 5.3 mg (30%) after RP-HPLC purification. Mw. calculated for C₄₆₈H₆₈₂N₄₈O₁₅₇S 9504.29. Found 9502.1 (MH⁺, MALDI-TOF MS).

### Example 17

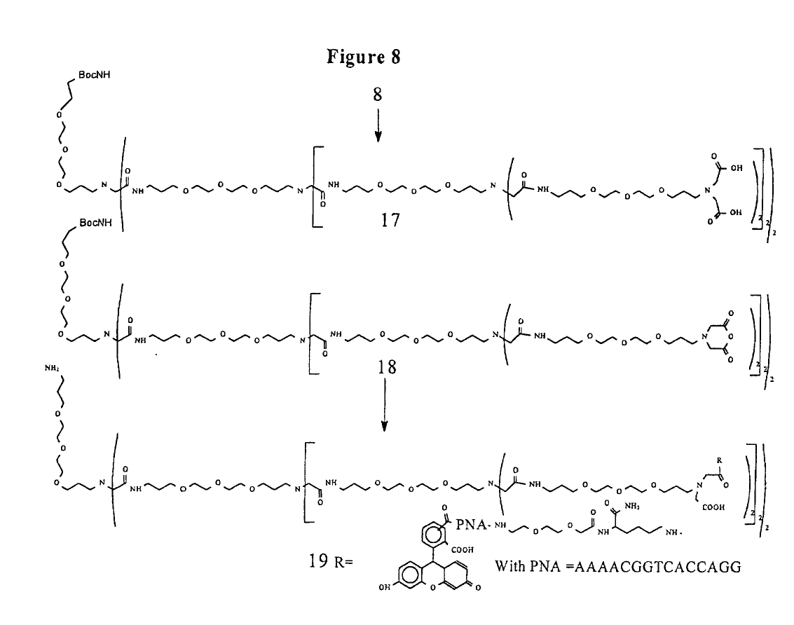

Preparation of 1-(tert-butoxycarbonyl)-1,15-diaza-4,7,10-trioxapentadecane-15-bis[3,17-diaza-2-oxo-7,10,13-trioxaheptadecane-17-bis(3,17-diaza-2-oxo-7,10,13-trioxaheptadecane-17-bis[3,17-diaza-2-oxo-7,10,13-trioxaheptadecane-17-bis(carboxymethyl)])], FIG. 8, No. 17

Partly protected dendrimer of the form Entity1-A-(free acetic acid)₁₆, dendritic core A=15 groups, each=—NH(CH₂)₃O(CH₂)₂O(CH₂)₂O(CH₂)₃N(CH₂C(O))₂—, Entity1=Boc, x1=1, dendritic core terminates in free acid form

The compound of Example 8 (100 mg) was dissolved in 2 ml methanol and 30 mg Pd/C was added. The mixture was hydrogenated under atmospheric pressure for 1 hour. The catalyst was filtered off, and the mixture reduced to dryness. Yield: 77 mg (100%). Mw. calculated for C₂₁₅H₃₈₄N₃₀O₈₅S 4749.3. Found 4752.3 (MH⁺, MALDI-TOF MS)

### Example 18

Preparation of 1-(tert-butoxycarbonyl)-1,15-diaza-4,7,10-trioxapentadecane-15-bis[3,17-diaza-2-oxo-7,10,13-trioxaheptadecane-17-bis(3,17-diaza-2-oxo-7,10,13-trioxaheptadecane-17-bis[3,17-diaza-2-oxo-7,10,13-trioxahexadecane-16-(2,6-dioxomorpholin-4-yl)])], FIG. 8, No. 18

Activated dendrimer of the form Entity1-A-(activated group)₈, dendritic core A=15 groups with 7 —NH(CH₂)₃O—(CH₂)₂O(CH₂)₂O(CH₂)₃N(CH₂C(O))₂—, and the peripheral 8=—NH(CH₂)₃O(CH₂)₂O(CH₂)₂O(CH₂)₃-(2,6-dioxomorpholine, Entity1=Boc, x1=1, dendrimer terminates in activated groups

The compound of Example 17 (10 mg) was dissolved in pyridine (100 μl) and diisopropylcarbodiimide (4 μl) was added. After 30 minutes at 55° C., the product was precipitated by addition of ether. Yield: 9 mg (91%). Mw. calculated for C₂₁₅H₃₈₄N₃₀O₈₅ 4749.34. Found 4751.2 (MH⁺, MALDI-TOF MS).

### Example 19

Preparation of a Dendrimer of the Form (Free Amine)-A-(Entity2)₈, FIG. 8, No.

Dendritic core A comprising 15 groups, 7 —NH(CH₂)₃O(CH₂)₂O—(CH₂)₂O(CH₂)₃N(CH₂C(O))₂—, the peripheral 8=—NH(CH₂)₃O—(CH₂)₂O(CH₂)₂O(CH₂)₃—(N(CH₂COOH)(CH₂C(O)—)), Entity2=peptide nucleic acid having the nucleobase sequence carboxyfluoresceinyl-AAAACGGTCACCAGG-linker-Lys-NH₂-wherein linker=—NH(CH₂)₂O(CH₂)₂OCH₂C(O)—, x2=8, Entity2 is coupled to A via the ε-N on Lys, dendritic core terminates in a free amine group

The peptide nucleic acid (carboxyfluoresceinyl-AAAACGGTCACCAGG-linker-Lys-NH₂) (2 mg) was dissolved in NMP (100 μl), TFA (20 μl) and DIPEA (50 μl). 0.2 mg of the compound of Example 18 was added and the mixture kept at 55° C. for 30 minutes. The crude product was precipitated by addition of ether, then suspended in TFA (100 μl) and re-precipitated. The product was purified by ultracentrifugation, the excess unreacted peptide nucleic acid passing through a 30 kDa cut-off filter. Yield: 1 mg (56%). Mw. calculated for C₁₇₅₄H₂₂₁₆N₇₉₈O₄₈₃ 42204.7. Found 42200 (MH⁺, MALDI-TOF MS).

### Example 20

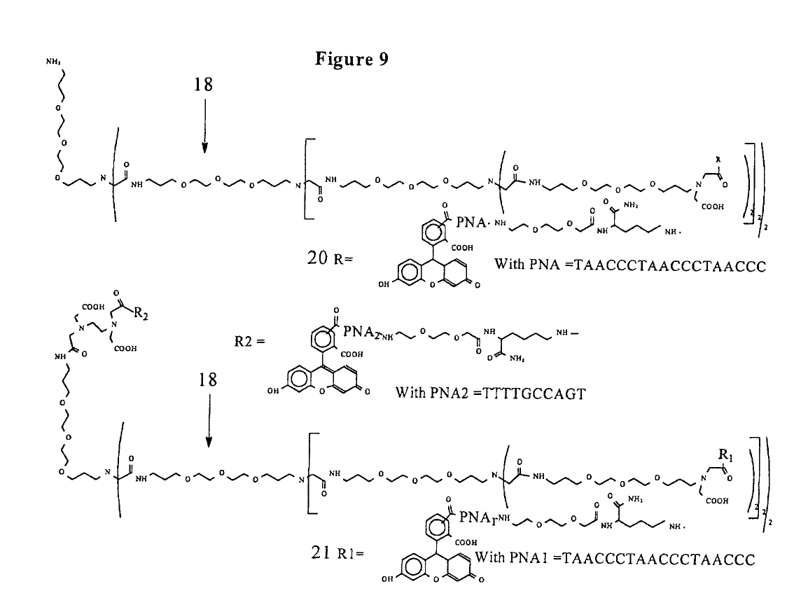

Preparation of a Dendrimer of the Form (Free Amine)-A-(Entity2)₈, FIG. 9, No. 20

Dendritic core A comprising 15 groups, 7 —NH(CH₂)₃O(CH₂)₂O—(CH₂)₂O(CH₂)₃N(CH₂C(O))₂—, the peripheral 8=—NH(CH₂)₃O—(CH₂)₂O(CH₂)₂O(CH₂)₃—(N(CH₂COOH)(CH₂C(O)—)), Entity2=peptide nucleic acid having the nucleobase sequence carboxyfluoresceinyl-TAACCCTAACCCTAACCC-linker-Lys-NH₂-wherein linker=—NH(CH₂)₂O(CH₂)₂OCH₂C(O)—, x2=8, Entity2 is coupled to A via the e-N on Lys, dendritic core terminates in a free amine

The peptide nucleic acid (carboxyfluoresceinyl-TAACCCTAACCCTAACCC-linker-Lys-NH₂) (1 mg) was dissolved in NMP (100 μl), TFA (20 μl) and DIPEA (50 μl). 0.1 mg of the compound of Example 18 was added and the mixture kept at 55° C. for 30 minutes. The crude product was precipitated by addition of ether, then suspended in TFA (100 μl) and re-precipitated. The product was purified by ultracentrifugation, the excess unreacted peptide nucleic acid passing through a 30 kDa cut-off filter. Yield: 0.6 mg (60%). Mw. calculated for C₁₉₈₆H₂₅₆₀N₈₅₄O₅₉₁ 47530.1. Found 47600 (MH⁺, MALDI-TOF MS)

### Example 21

Preparation of a Dendrimer of the Form (Entity1)-A-(Entity2)₈, FIG. 9, No. 21

Dendritic core A comprising 15 groups, 7=—NH(CH₂)₃O—(CH₂)₂O(CH₂)₂O(CH₂)₃N(CH₂C(O))₂, the peripheral 8 —NH(CH₂)₃O(CH₂)₂O(CH₂)₂O(CH₂)₃—(N(CH₂COOH)(CH₂C(O)—)) Entity1=carboxyfluoresceinyl-TTTTGCCAGT-Lys, Entity2=peptide nucleic acid having the nucleobase sequence carboxyfluoresceinyl-TAACCCTAACCCTAACCC-linker-Lys-NH₂-wherein linker=—NH(CH₂)₂O(CH₂)₂OCH₂C(O)—, and x1=1, x2=8, Entity1 is cross-linked to A via an EDTA, Entity2 is coupled to A via the ε-N on Lys

Excess EDTA dianhydride (1 mg) is reacted with carboxyfluoresceinyl-TTTTGCCAGT-Lys-NH₂ (1 mg) in NMP (100 μl), TFA (20 μl) and DIPEA (50 μl) to yield carboxyfluoresceinyl-TTTTGCCAGT-Lys(ε-N-EDTA monoanhydride)-NH₂ which is isolated by ether precipitation. This activated intermediary compound is re-dissolved in NMP (50 μl) and added to the dendrimer of Example 20 (0.6 mg) in 100 μl NMP, 20 μl TFA and 50 μl DIPEA at 55° C. After 1 hour, the product is precipitated with ether. The product was purified by ultracentrifugation, the excess activated intermediary compound passing through a 30 kDa cut-off filter. Yield: 0.3 mg (46%). Mw. calculated for C₂₁₃₁H₂₇₃₀N₉₀₉O₇₁₈ 50966.9. Found 51100 (MH⁺, MALDI-TOF MS).

### Example 22

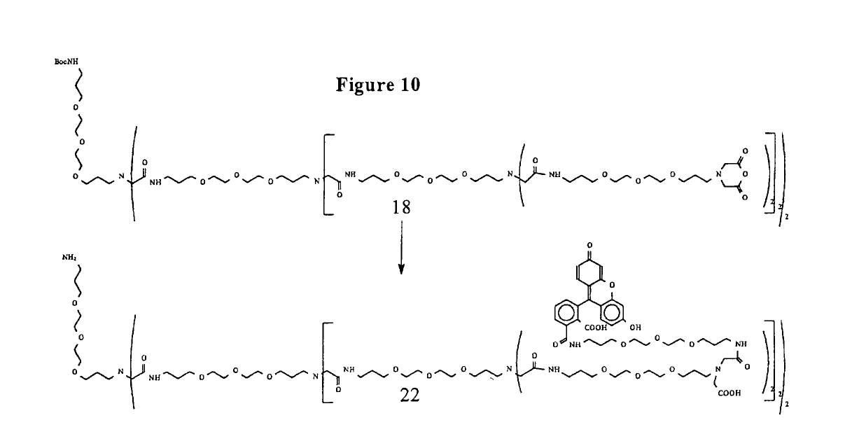

Preparation of a Dendrimer of the Form (Free Amine)-A-(Entity2)₈, FIG. 10, No. 22

Dendritic core A comprising 15 groups, 7=—NH(CH₂)₃O—(CH₂)₂O(CH₂)₂O(CH₂)₃N(CH₂C(O))₂—, the peripheral 8=—NH(CH₂)₃O(CH₂)₂O(CH₂)₂O(CH₂)₃—(N(CH₂COOH)(CH₂C(O)—)), Entity2=linker-carboxyfluorescein wherein linker=—NH(CH₂)₃O—(CH₂)₂O(CH₂)₂O(CH₂)₃NH—, x2=8

The compound of Example 18 (10 mg) was dissolved in NMP (100 μl), and DIPEA (20 μl) and (13-carboxyfluoresceinylamido)-4,7,10-trioxotridecane-1-amine (13 mg) were added. After 10 minutes at 55° C., the crude product was isolated by precipitation with ether and subsequently dissolved in 100 μl TFA and re-precipitated with ether. Yield: 7 mg (36%) after RP-HPLC purification. Mw. calculated for C₄₅₈H₆₄₈N₄₆O₁₅₅ 9278.4. Found 9281.5 (MH⁺, MALDI-TOF MS).

### Example 23

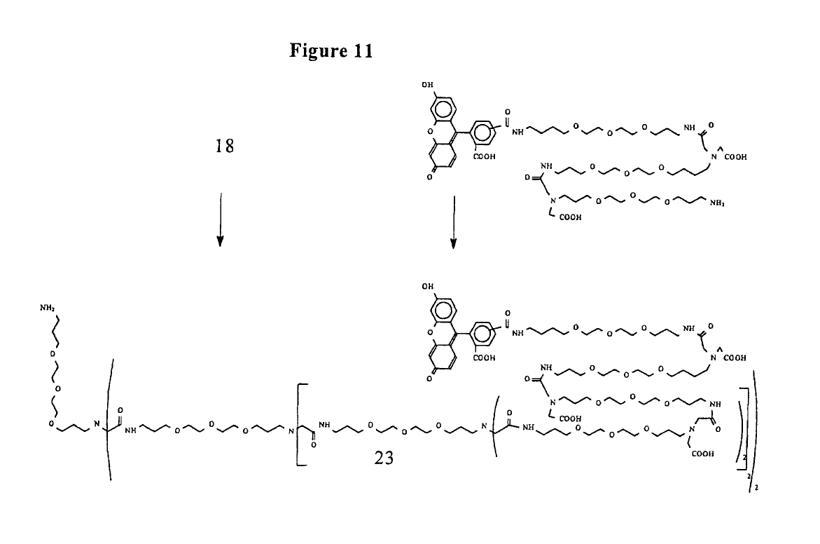

Preparation of Dendrimer of the Formula (Free Amine)-A-(Entity2)₈, FIG. 11, No. 23

Dendrimer wherein the dendritic core A comprises 15 groups, 7=—NH(CH₂)₃O(CH₂)₂O(CH₂)₂O(CH₂)₃N(CH₂C(O))₂—,—, the peripheral 8=—NH(CH₂)₃O(CH₂)₂O(CH₂)₂O(CH₂)₃—(N(CH₂COOH)(CH₂C(O)—)), Entity2=AAL-AAL-linker-carboxyfluorescein wherein linker=—NH(CH₂)₃O(CH₂)₂O(CH₂)₂O—(CH₂)₃NH—, and AAL=—NH(CH₂)₃O(CH₂)₂O(CH₂)₂O(CH₂)₃—(N(CH₂COOH)(CH₂C(O)—)), x2=8, dendrimer terminates in a free amine

(13-Carboxyfluoresceinylamido)-4,7,10-trioxotridecane-1-amine, 57 mg, was dissolved in 0.5 mL NMP and 0.1 mL DIPEA. The compound of Example 31, 50 mg in 250 μL pyridine, was added. After 5 min the intermediate product was precipitated with ether, deprotected with 0.5 mL TFA, precipitated, neutralised with 0.1 mL DIPEA and dissolved in 0.5 mL NMP. It was again reacted with 75 mg of compound 31 in 375 μL pyridine for 5 min, followed by precipitation and 5 min TFA deprotection. Finally HPLC purification afforded 25 mg of AAL-AAL-linker-carboxyfluorescein. MALDI-TOF MS: Found 1214. Calculated 1214.1 (M⁺H). This was reacted with the compound of Example 18 (10 mg) in NMP (100 μL) and DIPEA (30 μL) for 10 min at room temperature. Precipitation with ether followed by by final TFA deprotection and HPLC purification gave 15 mg of the title compound. MALDI-TOF MS: Found 14460. Calculated 14445.

### Example 24

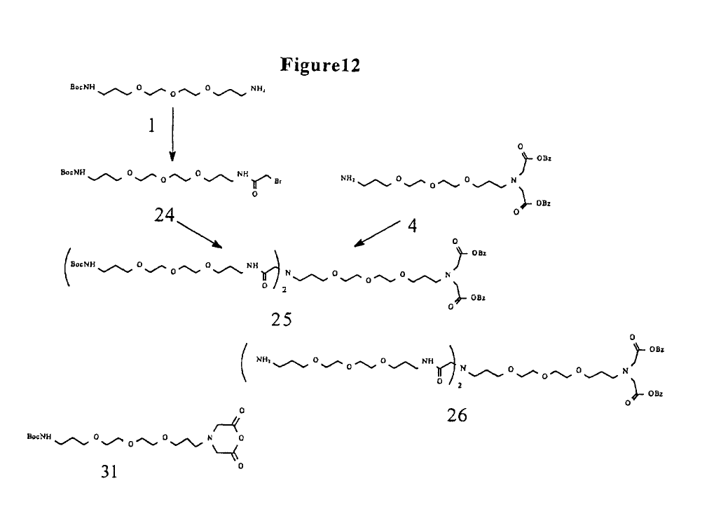

Preparation of N-Boc,N′(bromoacetyl)-4,7,10-trioxotridecanediamine, FIG. 12, No. 24

The compound of Example 1, 16 g, 50 mmol, was dissolved in 50 mL DCM and added dropwise over 30 min to 55 mmol bromoacetic anhydride and 70 mmol lutidin in 200 mL ice cold DCM. The mixture was extracted twice with 100 mL 1 M citrate pH 4.5, reduced to partial dryness, and immidiately purified with 5% MeOH in DCM. Yield 17.6 g, 80%. MALDI-TOF MS (M⁺H-Boc): Found 342.2. Calculated 343.

### Example 25

Preparation of DiAminoAcidLinker-dibenzyl Ester (DAAL-dibenzyl Ester)

Dendrimer of the formula (Entity1)₂-A-(Entity2)₂, wherein Entity1=Boc, Entity2=OBz, x1=1, x2=2, and A=DAAL

The compound of Example 4, 5.15 g free amine form, 10 mmol, was dissolved in 30 mL DMF and 15 mL DIPEA. N-Boc,N′(bromoacetyl)-4,7,10-trioxotridecanediamine, 10 g, was added and the mixture stirred for 2 h at 50 C. The solvent was removed under reduced pressure, the mixture taken up in 100 mL DCM and extracted with 100 mL sodium hydrogen carbonate, then 100 mL water. Reduction to dryness and subsequent chromatography with 5-15% MeOH in DCM gave 6.8 g, 55%, as a viscous oil. MALDI-TOF MS (M³⁰ H): Found 1237.3. Calculated 1238.2.

### Example 26

Preparation of DiAminoAcidLinker-dibenzyl Ester (DAAL-dibenzyl Ester), Free Amine Form, FIG. 12, No. 26

Dendrimer of the formula (free amine)₂-A-(Entity2)₂, wherein Entity2=OBz, x2=2, and A=DAAL

DiAminoAcidLinker-dibenzyl ester, 6.8 g, was dissolved in 30 mL TFA. After 5 min TFA was removed under reduced pressure, and the mixture taken up in 200 mL DCM and extracted twice with 150 mL sodium carbonate. The organic phase was reduced to dryness and further dried by evaporation from dry toluene under reduced pressure. Yield 4.8 g, 84%. MALDI-TOF MS: Found 1038.6. Calculated 1038.1.

### Example 27

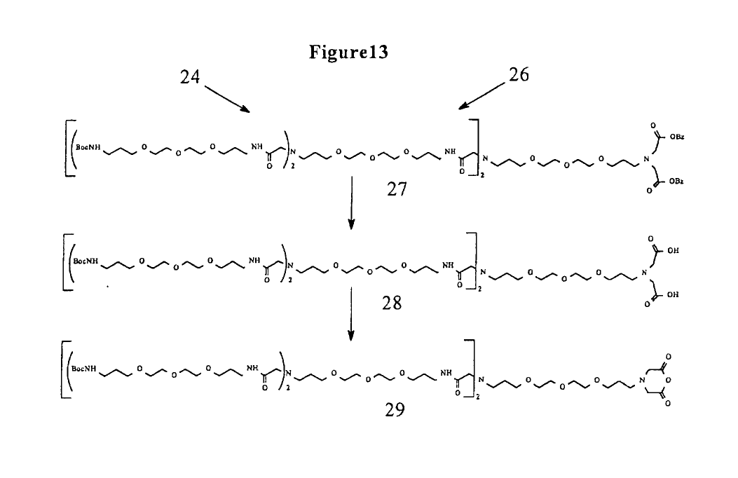

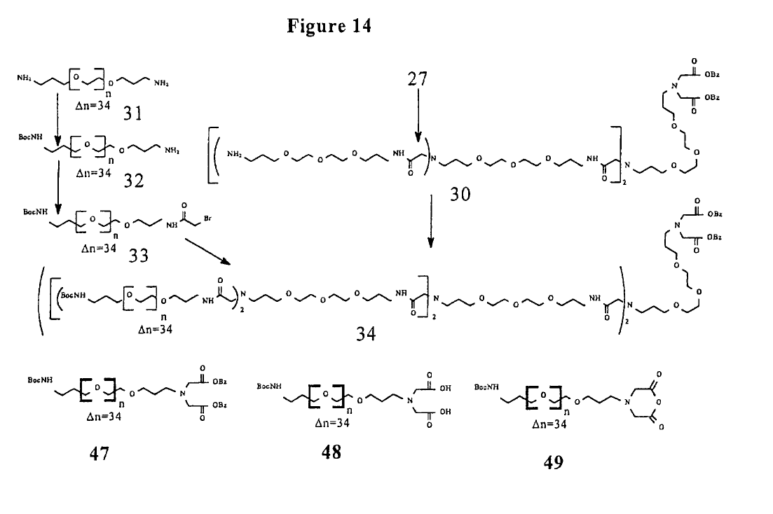

Preparation of TetraAminoAcidLinker-dibenzyl Ester (TAAL-dibenzyl Ester), FIG. 13, No. 27

Dendrimer of formula (Entity1)₄-A-(Entity2)₂, wherein Entity1=Boc, Entity2=OBz, A=3 —NH(CH₂)₃O—(CH₂)₂O(CH₂)₂O(CH₂)₃NHC(O)CH₂—, and 2 —NH(CH₂)₃O—(CH₂)₂O(CH₂)₂O(CH₂)₃N(CH₂C(O)—)₂

The compound of Example 26, 4.8 g, was dissolved in 20 mL DMF and 10 ML DIPEA. 10 g of N-Boc,N′(bromoacetyl)-4,7,10-trioxotridecanediamine was added and the mixture stirred for 4 h at 50° C. Removal of the solvent under reduced pressure gave an oil that was taken up in 100 mL DCM and extracted with sodiumhydrogencarbonate, followed water. The organic phase was removed, and the residue chromatographed on silica with first 15% MeOH in ethylacetate, and subsequently eluded with 15% MeOH in DCM. The so obtained product was purified on silica with 10% ammonia saturated MeOH in DCM to give 2.2 g, 19%, of a viscous clear oil. MALDI-TOF MS: Found 2480. Calculated 2479.

### Example 28

Preparation of TetraAminoAcidLinker (TAAL), Free Diacid Form, FIG. 13, No. 28

Dendrimer of the form (Entity1)₄-A-(free acetic acid)₂, wherein Entity1=Boc, x1=4, A=TAAL

The TetraAminoAcidLinker-dibenzyl ester of Example 27, 1.2 g, was dissolved in 20 mL MeOH, 200 mg 10%Pd/C was added, and the mixture hydrogenated for 2 h. Catalyst and solvent were removed by centrifugation and subsequent evaporation to give 1 g of the product. MALDI-TOF MS: Found 2300. Calculated 2299.

### Example 29

Preparation of TetraAminoAcidLinker-anhydride, FIG. 13, No. 29

Dendrimer of the structure (Entity1)₄-A-(N-2,6-dioxomorpholine), wherein Entity1=Boc, x1=4, A=3 —NH(CH₂)₃O—(CH₂)₂O(CH₂)₂O(CH₂)₃NHC(O)CH₂—, dendrimer terminates a N-2,6-dioxomorpholine group TetraAminoAcidLinker, free diacid form, of Example 27, 100 mg was dissolved in 1 mL pyridine. 0.1 mL diisopropylcarbodiimide was added and the mixture reacted at 70° C. for 10 min. This approx 0.38 M solution of the product was used immediately without further purification. Reaction of such solution with excess free primary amine under basic conditions indicated >90% conversion to anhydride.

### Example 30

Preparation of TetraAminoAcidLinker-dibenzylester-free Tetraamine

Dendrimer of the structure (free amine)₄-A-(Entity2)₂, wherein Entity2=OBz, X2=2, A=3 —NH(CH₂)₃O—(CH₂)₂O(CH₂)₂O(CH₂)₃NHC(O)CH₂—, and 2 —NH(CH₂)₃O—(CH₂)₂O—(CH₂)₂O(CH₂)₃N(CH₂C(O)—)₂, dendritic core terminates in a free amine

TetraAminoAcidLinker-dibenzyl ester, 230 mg, 0.1 mmol, was dissolved in 1 mL TFA. After 5 min, 7 mL ether was added, and the product isolated by centrifugation. It was repeatedly votexed with ether to afford a 300 mg of a semisolid TFA-adduct, that was used immediately without further purification. MALDI-TOF MS: Found 2078. Calculated 2079.

### Example 31

Preparation of AminoAcidLinker-anhydride (AAL-anhydride)

The compound of Example 3, 53 mg in 0.25 mL pyridine, was activated with 25 μL diisopropylcarbodiimide for 10 min at 70° C., and used in solution without further purification. The yield was assumed to be quantitative, 50 mg.

### Example 32

Preparation of O-(Boc-aminopropyl)-O′-aminopropyl-ethyleneglycol (Δ 34 glycol Units)

O,O′-Bisaminopropyl-ethylenglycol was dissolved in 250 mL THF. The mixture was cooled on an ice bath and 1,18 g Boc-anhydride in 15 mL THF was added drop-wise, and the mixture reduced to 20 g of a viscous oil. This oil was purified on silica with 20% MeOH in DCM, then eluded with 20% half ammonia saturated MeOH in DCM. The product was re-purified with 15% half ammonia saturated MeOH in DCM to give 2.75 g, 29%. MALDI-TOF MS: Found (44.05)n+235. Calculated (44.05)n+233.

### Example 33

Preparation of O-(Boc-aminopropyl)-O′-(bromoacetamidopropyl)-ethyleneglycol (Δ 34 Glycol Units)

O-(Boc-aminopropyl)-O'aminopropyl-ethyleneglycol (Δ 34 glycol units), 1.1 g, was dissolved in 3 mL DCM and added to 0.7 mmol bromoaceticanhydride and 1 mmol lutidine in 3 mL ice cold DCM. After 15 min reaction the mixture was extracted twice with 2 mL 1 M sodium citrate, pH 4.5, and following reduction to dryness, it was purified on silica with 15% MeOH in DCM. Yield 1 g, 85%. MALDI-TOF MS (M-Boc) Found (44.05)n+252. Calculated (44.05)n+253.

### Example 34

Preparation of Octa-(Boc-amino-PEG)-dendrimer-dibenzylester

Protected dendrimer of form (Entity1)₈-A-(Entity2)₂ wherein Entity1=Boc, Entity2=OBz, x1=8, x2=2, A=3 —NH(CH₂)₃O—(CH₂)₂O(CH₂)₂O(CH₂)₃NHC(O)CH₂—, and 2 —NH(CH₂)₃O—(CH₂)₂O(CH₂)₂O(CH₂)₃N(CH₂C(O)—)₂, and 8 —NH(CH₂)₃O(CH₂CH₂O)₃₄(CH₃)NH—

TetraAminoAcidLinker-dibenzylester-free tetraamine TFA adduct, 100 mg, approximately 0.03 mmol, was dissolved in 1 mL DMF and 0.5 mL DIPEA. O-(Boc-aminopropyl)-O′-(bromacetamidopropyl)-ethyleneglycol (Δ 34 glycol units) 1 g, approximately 0.5 mmol, was added and the mixture stirred for 4 h at 60° C. 5 mL DCM was added and the mixture extracted with 2 mL sodium hydrogen carbonate, then with 1 mL water. DCM was evaporated off, and the product precipitated by addition of 12 mL ether. Yield 400 mg. MALDI-TOF MS: Found: 16600 (8 PEG-arms) and as a major impurity 12900 (6 PEG-arms).

### Example 35

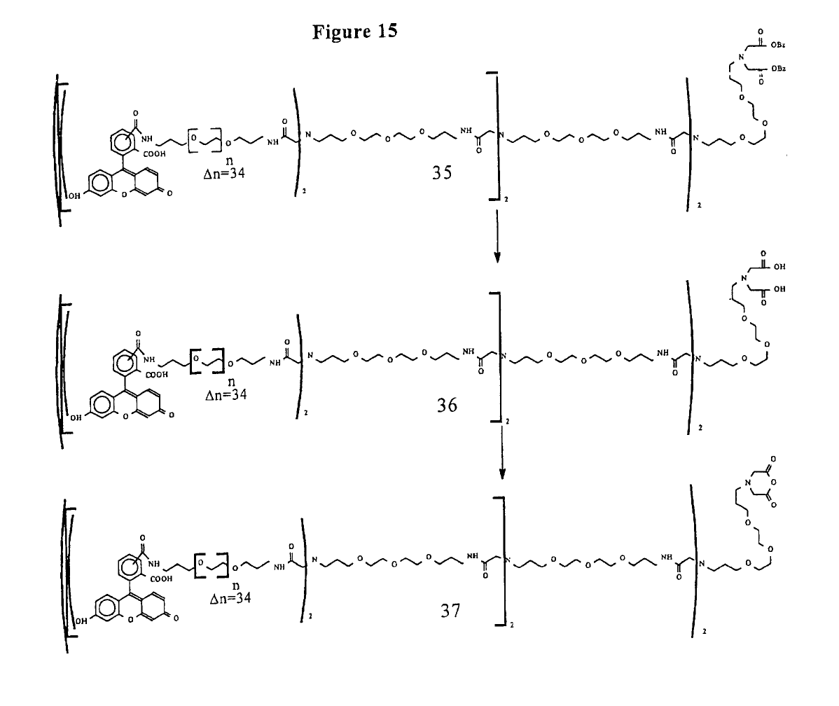

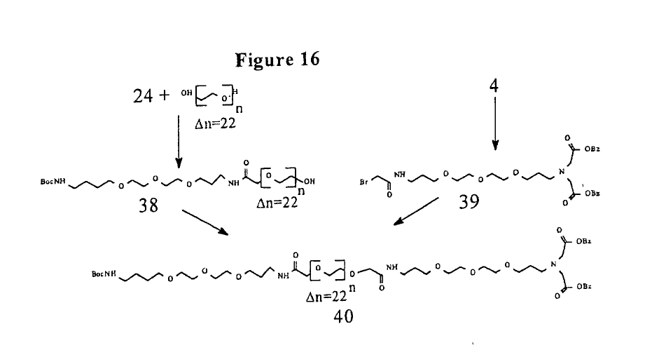

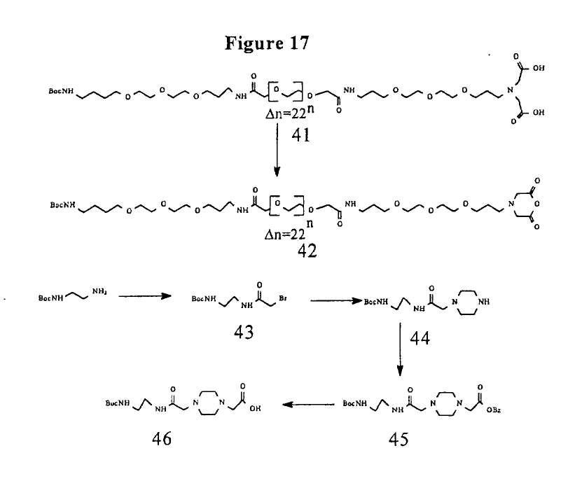

Preparation of Octa-(carboxyfluoresceinamido-PEG)-dendrimer-dibenzylester, FIG. 15, No. 35

Dendrimer of the form (Entity1)₈-A-Entity2, wherein Entity1=Fluorescinyl, Entity2=OBz, x1=8, x2=1, A=3 —NH(CH₂)₃O—(CH₂)₂O(CH₂)₂O(CH₂)₃NHC(O)CH₂—, and 2NH(CH₂)₃O—(CH₂)₂O(CH₂)₂O(CH₂)₃N(CH₂C(O)—)₂, and 8 NH(CH₂)₃O(CH₂CH₂O)₃₄(CH₃)NH—

Octa-(Boc-amino-PEG)-dendrimer-dibenzylester, 200 mg, was dissolved in 1 mL TFA and after 5 min the intermediate free octa-amine was precipitated by addition of 7 mL ether. It was vortexed with ether, then dissolved in 1 mL NMP and 0.3 mL DIPEA. N-hydroxy-succinimido-carboxy-fluorescein, 50 mg, was added, and after 10 min additional 50 mg was added. After 30 min in all, the product was precipitated by addition of 7 mL ether. MALDI-TOF MS: Found: 18600 (major product, consistent with 8 fluorescinylated PEG arms).

### Example 36

Preparation of Octa-(carboxyfluoresceinamido-PEG)-dendrimer-free Diacid, FIG. 15, No. 36

Dendrimer of the form (Entity1)₈-A-(free acid)₂ Entity1=Fluorescinyl, x1=8, A=3 —NH(CH₂)₃O—(CH₂)₂(CH₂)₂O(CH₂)₃NHC(O)CH₂—, and 2NH(CH₂)₃O—(CH₂)₂O(CH₂)₂O(CH₂)₃N(CH₂C(O))₂, and 8 NH(CH₂)₃O(CH₂CH₂O)₃₄(CH₃)NH—

Octa-(carboxyfluoresceinamido-PEG)-dendrimer-dibenzyl ester of Example 35, approximately 100 mg, was dissolved in 1 mL 1.0 M LiOH and 2 mL THF. After 30 min reaction, 1 mL 1.0 M HCl was added. Further 6 mL of water was added, and addition of ammonia to pH 8 afforded an almost black (intensely red) but clear solution. RP-HPLC allowed isolation of the title compound having 8 fluorescein groups in the outermost layer (eluding with approximately 55% acetonitrile in 0.1% aqueous TFA) in 18 mg yield. MALDI-TOF MS: Found 18400. Further 30 mg of dendrimers with 6 and 7 fluoresceinylated arms were also isolated.

### Example 37

Preparation of Octa-(carboxyfluoresceinamido-PEG)-dendrimer-anhydride, FIG. 15, No. 37

Dendrimer of the form (Entity1)₈-A-(N-2,6-dioxomorpholine)₂,

Entity 1=fluorescinyl, x1=8,

A=1N(CH₃)₂O(CH₂)₂O(CH₂)₂O(CH₃)₃,

3N(CH₃)₂O(CH₂)₂O(CH₂)₂O(CH₃)₃NHC(O)CH₂,

8NH(CH₂)₃O(CH₂CH₂O)₃₄(CH₂)₃NHC(O)CH₂, dendrimer terminates in dioxomorpholine groups

Octa-(carboxyfluoresceinamido-PEG)-dendrimer-free diacid, 3 mg in 50 μL NMP, was reacted with 5 μL diisopropylcarbodiimide for 15 min at room temperature and precipitated by addition of 1 mL ether. It was used without further purification. A control reaction with a large excess of the compound of Example 32 (O-(Boc-aminopropyl)-O′aminopropyl-ethyleneglycol (Δ 34 glycol units)) indicated that approximately 85% of the product was activated this way.

### Example 38

Preparation of Boc-amino PEG

50 g of PEG 1000, 50 mmol, was dissolved in 300 mL THF and 10 mL 2M, butyllilium was added. The compound of Example 24, 9 g in 30 mL THF, was added and the solution stirred overnight under nitrogen. Reduction to dryness, extraction with hydrogen carbonate from DCM and repeated chromatography with methanol in DCM afforded 12 g 50%. MALDI-TOF MS: Found (44.053)n

### Example 39

Preparation of 1-(bromoacetyl)-1,15-diaza-4,7-10-trioxapentadecane-15-bis(benzyloxycarbonylmethyl)

Dendrimer of the form (Entity 1)ₓ₁-A-(Entity 2)ₓ₂, Entity 1=bromoacetyl, x1=1, Entity 2 O-Bz, x2=2 A=1NH(CH₃)₂O(CH₂)₂O(CH₂)₂O(CH₃)₃N((CH₂)C(O))₂

The TFA adduct of Example 4 was extracted with hydrogen carbonate from DCM and dried by evaporation from toluene under reduced pressure, to give the free amine in 80% yield. 10.3 g, 20 mmol of this product was dissolved in 25 mL toluene and added drop-wise over 30 min to 22 mmol bromoacetic anhydride and 30 mmol lutidin in 100 mL ice cold DCM. The mixture was extracted twice with 100 mL 1 M citrate pH 4.5, reduced to partial dryness, and immediately purified with 5% MeOH in DCM (r.f 0.8). Yield 80%. MALDI-TOF MS: Found 638.7. Calculated 638.

### Example 40

Preparation of Boc-PEG-linker Dibenzylester

Dendrimer of the form (Entity1)ₓ₁-A-(Entity2)ₓ₂, wherein Entity1=Boc, Entity2=OBz, x1=1, x2=2, A=—NH(CH₂)₃O(CH₂)₂O(CH₂)₂O(CH₂)₃NHC(O)CH₂(O(CH₂)₂)₂₂OCH₂C(O)—NH(CH₂)₃O(CH₂)₂O(CH₂)₂O(CH₂)₃N(CH₂C(O))₂

Boc-amino-PEG of Example 38, 12 g, was dissolved in 60 mL DCM and 5.5 mL 2 M butyllithium was added. With cooling 4.4 g of 1-(bromoacetyl) 1,15-diaza-4,7-10-trioxapentadecane-15-bis(benzyloxycarbonylmethyl) in 10 mL THF was added and the mixture stirred overnight at room temperature. Reduction to dryness, extraction with hydrogen carbonate and repeated chromatographic work-up afforded 1.7 g 10%. MALDI-TOF MS:

### Example 41

Preparation of Boc-PEG-linker Free Diacid

Dendrimer of the form (Entity1)ₓ₁-A wherein Entity1=Boc, x 1=1, A=NH(CH₂)₃O(CH₂)₂O(CH₂)₂O(CH₂)₃NHC(O)CH₂(O(CH₂)₂)₂₂OCH₂C(O)—NH(CH₂)₃O(CH₂)₂(CH₂)₂O(CH₂)₃N(CH₂C(O))₂ Dendrimer terminates in free acids

Boc-PEG-linker dibenzylester of Example 40, 1.7 g, 1 mmol, was dissolved in 15 mL MeOH. Under nitrogen 300 mg 10% Pd/C was added and the mixture hydrogenated for 2 h. Removal of catalyst and solvent gave a quantitative yield of a viscous oil. This compound was stored as a 20% solution in pyridine, for subsequent reaction.

### Example 42

Preparation of Boc-Peg-linker Anhydride

Boc-PEG-linker free diacid, 20% in pyridine, was activated with 10% diisopropylcarbodiimide for 10 min at 70° C. to afford an approx. 1M solution of the anhydride which was used fresh.

### Example 43

Preparation of N-Boc-N′-bromoacetyl-ethylendiamine

16 g mono-Boc-ethylenediamine, 100 mmol, was dissolved in 50 mL DCM and added drop-wise over 30 min to 110 mmol bromoacetic anhydride and 130 mmol lutidin in 350 mL ice cold DCM. The mixture was filtered and extracted twice with 200 mL 1 M citrate pH 4.5, filtered, reduced to partial dryness and stirred on an ice bath with 150 mL ether. The product was filtered off and dried. Yield 10 g, light tan powder.

### Example 44

Preparation of N-Boc-N′-(N-piperazinyl-acetyl)-ethylenediamine

4.3 g piperazine was dissolved in 50 mL THF and 2.8 g N-Boc-N′-bromoacetyl-ethylendiamine was added. After 1 h the mixture was reduced to dryness and taken up in 50 ml DCM and extracted once with water. Reduction to dryness and chromatographic workup with 10% ammonia saturated MeOH in DCM gave 1.4 g, 50% as an oil that slowly solidified

### Example 45

Preparation of Boc-piperazine-linker Benzylester

N-Boc-N′-(N-piperazinyl-acetyl)-ethylenediamine, 1.4 g, was dissolved in 5 mL DMF and 1 mL DIPEA, 1.5 mL was added, followed by 1.2 g bromoacetic acid benzylester. The mixture was stirred for 30 min at 40° C., taken up in 50 mL DCM and extracted with 50 mL sodium hydrogen carbonate. Chromatography with 5% MeOH in DCM afforded 800 mg, 36%. MALDI-TOF MS (M⁺H): Found 438.7. Calculated 439.4.

### Example 46

Preparation of Boc-piperazine-linker, Free Acid

Boc-piperazine-linker benzylester, 800 mg, was dissolved in 10 mL MeOH. Under nitrogen 100 mg 10%Pd/C was added and the mixture hydrogenated for 1 h. Pd/C was removed by centrifugation, and subsequent evaporation of the solven afforded the title compound in quantitative yield, 620 mg. MALDI-TOF MS (M⁺H): Found 347.1. Calculated 347.4.

### Example 47

Preparation of PEG34-linker-dibenzylester

Dendrimer of the form (Entity1)ₓ₁-A-(Entity2)ₓ₂, wherein Entity1=Boc, Entity2=OBz, x1=1, x2=2, A=—NH(CH₂)₃(O(CH₂)₂)₃₄O(CH₂)₃N(CH₂C(O))₂

O-(Boc-aminopropyl)-O′aminopropyl-ethyleneglycol (Δ 34 glycol units), 1.49 g, was dissolved in 5 ml DMF and 1.5 mL DIPEA. 616 μL bromoaceticacid benzylester was added and the mixture reacted for 1 h at 60° C. The solvent was removed, and the residue taken up in 25 mL DCM. It was extracted with 25 mL sodium hydrogen carbonate, which was in turn back-extracted with 25 mL DCM. The combined organic phases were reduced and purified on silica with 10% MeOH in DCM, eluding with 15% MeOH in DCM. Yield 810 mg, 46% as a clear viscous oil. MALDI-TOF MS (M-Boc): Found (44.05)n+429. Calculated (44.05)+430.

### Example 48

Preparation of PEG34-linker-free Diacid

Dendrimer of the form (Entity1)ₓ₁-A, wherein Entity1=Boc, x1=1, A=—NH(CH₂)₃(O(CH₂)₂)₃₄O(CH₂)₃N(CH₂C(O))₂ dendrimer terminates in free acids.

PEG34-linker-dibenzylester, 0.81 g, was dissolved in 20 mL MeOH and 160 mg Pd/C was added. The mixture was hydrogenated for 2 h, and following removal of Pd/C and solvent, 723 mg of the free diacid was obtained (98%).

### Example 49

Preparation of 49 PEG34-linker-anhydride

95.6 μl of a 20% solution of PEG34-linker-free diacid in pyridine was reacted with 9.65 μL diisopropyldiiminde for 10 min at 70° C. and used without further purification.

### Example 50

Preparation of Dendrimer of the Formula ((FluPEG)₄TAAL)₄TAAL-PEG-PEG-PNA, Wherein Entity1=FluPEG, x1=16, Entity 2=PNA, Wherein the Peptide Nucleic Acid has the Nucleobase Sequence TGT GCG CCC TCA ACT AAC, x2=1

This procedure was also used for preparing the compounds of Examples 50-57.

The piperazine linker of Example 46 was directly coupled to the PNAs using standard solid phase Boc chemistries.

The other linkers were coupled in solution to the PNAs and derivatives using the following general procedure: The PNA was dissolved in NMP to a concentration of 1-5 mM. If this could not be achieved in neat NMP, addition of 20% water resulted in a clear solution of desired concentration. Residual TFA from the HPLC purification of the PNAs was then neutralised by addition of 5% pyridine. 2 equivalents of the appropriate Boc-protected linker-anhydride was added, followed by addition of 20% DIPEA. The reaction was followed by MALDI, in case of incomplete reaction after 10 min (observed mainly when using water as co-solvent due to competing hydrolysis), additional 2 equivalents of linker were added.

The crude product was precipitated by addition of lOx excess of ether.

Boc-deprotection was achieved by dissolving in 50-100×excess 5% meta-cresol in TFA for 10 min, followed by precipitation with further 10×excess ether.

Either additional linkers were attached to the PNA-constructs, if necessary following HPLC purification, or the constructs were labelled using commercially available amino-reactive labels in highest possible concentration, typically 3-15 weight % label, typically 3-30×molar excess of label relative to free amino groups.

### Example 51

Preparation of Dendrimer of the Formula ((FluPEG)₄TAAL-PEG)₄TAAL-PEG-PNA, Wherein Entity1=FluPEG, x1=16, Entity 2=PNA, Wherein the Peptide Nucleic Acid has the Nucleobase Sequence TGT GCG CCC TCA ACT AAC, and x2=1

The product was prepared according to the procedure described in Example 50.

### Example 52

Preparation of a Dendrimer of Formula (FluPEG)₄TAAL-PNA-Lys-Cy3, Wherein Entity1=FluPEG, x1=4, Entity2=Cy3, x2=1, Lys=Lysine, and Wherein PNA is a Peptide Nucleic Acid with the Nucleobase Sequence TGT GCG CCC TCA ACT AAC

The product was prepared according to the procedure described in Example 50.

### Example 53

Preparation of a Dendrimer Complex of Formula (label-PEG)₄TAAL-PNA-Lys-TAAL(PEG-label)₄, Wherein Entity1=label-PEG, the Label Being Flu (Fluorescein), Rho (Rhodamine) Lis (Lissamine), Cy3, or DNP, x1=4, Dendritic Core A=TAAL Coupled to PNA-Lys Coupled to Another Dendritic Core A=TAAL, Entity1=PEG-Label, x1=4, and Wherein the Peptide Nucleic Acid, PNA, has the Nucleobase Sequence TGT GCG CCC TCA ACT AAC

The product was prepared according to the procedure described in Example 50.

A: Label being Flu

B: Label being Rho

C: Label being Lis

D: Label being Cy3

E: Label being DNP

### Example 54

Preparation of Dendrimer Complex of Formula ((FluPEG)₄TAAL)₄TAAL-PNALys-TAAL(TAAL)₄(PEGFlu)₄), Wherein Entity1=FluPEG, x1=16, Dendritic Core Coupled to PNA-Lys Coupled to Another Dendritic Core, Wherein Entity1=FluPEG, x1=16, and Wherein the Peptide Nucleic Acid PNA has the Nucleic Acid Sequence A: CCA TAT GCA GTT ATA AGT AGG, B: TAT TGT ACC AAG CAG AGT ACC, C: GGT ATA TAT AAG ATG ACA CAG GA, and D: GTT AGT TAT ATT GGG TGA TAT GT

The product was prepared according to the procedure described in Example 50.

### Example 55

Preparation of Dendrimer Complex of the Formula (Flu(AAL)₂)TAAL-PNALys-TAAL((ALL)₂Flu)₄, Wherein Entity1=Flu(AAL)₂, x1=4, Dendritic Core Coupled to PNA-Lys Coupled to Another Dendritic Core, Wherein Entity1=Flu(AAL)₂, x1=4, Wherein the Peptide Nucleic Acid PNA has the Nucleic Acid Sequence A: CCA TAT GCA GTT ATA AGT AGG, B: TAT TGT ACC AAG CAG AGT ACC, C: GGT ATA TAT AAG ATG ACA CAG GA, and D: GTT AGT TAT ATT GGG TGA TAT GT

The product was prepared according to the procedure described in Example 50.

### Example 56

Preparation of Dendrimer Complex of the Formula (Label-PEG)₄TAAL-PNALys-TAAL(PEG-label)₄, wherein Entity1=labelPEG, x1=4, Dendritic Core Coupled to PNA-Lys Coupled to Another Dendritic Core, Wherein Entity1 labelPEG, x1=4, and Wherein the Peptide Nucleic Acid PNA has the Nucleic Acid Sequence A: CCA TAT GCA GTT ATA AGT AGG, B: TAT TGT ACC AAG CAG AGT ACC, C: GGT ATA TAT AAC ATG ACA CAG GA, and D: GTT AGT TAT ATT GGG TGA TAT GT

The product was prepared according to the procedure described in Example 50.

### Example 57

Preparation of a Dendrimer Complex of the Formula (FluPEG)₄TAAL-PIP-PIP-PNA-PIP-PIP-Lys-TAAL (FluPEG)₄, Wherein Entity1=FluPEG, x1=4, Dendritic Core A Coupled to PIP-PIP-PNA-PIP-PIP-Lys Coupled to a Dendritic Core A, Wherein Entity1=FluPEG, x1=4, and Wherein the Peptide Nucleic Acid, PNA, has the Nucleobase Sequence TGT GCG CCC TCA ACT AAC

The product was prepared according to the procedure described in Example 50.

### Example 58

Preparation of a Dendrimer Complex of Formula (Flu-PEG)₄TAAL-PNA-Lys-TAAL(PEG-Flu)₄, Wherein Entity1=Label-PEG, x1=4, Dendritic Core A=TAAL Coupled to PNA-Lys Coupled to Another Dendritic Core A=TAAL, Entity1=PEG-Label, x1=4,, and Wherein the Peptide Nucleic Acid, PNA, has the Nucleobase Sequence A: TuGTu GCG CCC TUCD DCT DDC

The product was prepared according to the procedure described in Example 50.

### Example 59

Preparation of a Dendrimer Complex of Formula (Rho-PEG)₄TAAL-PNA-Lys-TAAL(PEG-Rho)₄, Wherein Entity1=label-PEG, x1=4, Dendritic Core A=TAAL Coupled to PNA-Lys Coupled to Another Dendritic Core A=TAAL, Entity1=PEG-Label, x1=4,, and Wherein the Peptide Nucleic Acid, PNA, has the Nucleobase Sequence GTuTu DGTu TuGD GGG CGC DCD

The product was prepared according to the procedure described in Example 50.

### Example 60

Evaluation of the Probes

The experiment was carried out on metaphase chromosome spreads using the dendrimer complexes of expamles 50-59 and single-labelled conventional peptide nucleic acid probes having a corresponding nucleobase sequence. The samples were denatured in 60% formamide at 80° C. for 10 minutes. Then the dendrimer complexes or the single-labelled probes in hybridisation buffer (as defined in Example 65) was added, and the slides were allowed to cool to room temperature, following stringent wash at 55° C. The probes were scored on a scale from 1 to 5, the signal obtained from the single-labelled set to 2.

| Score | Dendrimer or dendrimer complex |
|---|---|
| 2 | Single-labelled probe (2,6) |
| 3 | Example 50 |
| 2 | Example 51 |
| 3 | Example 52 |
| 3 | Example 53 A |
| 3 | Example 53 B |
| 3 | Example 53 C |
| 4 | Example 53 D |
| 4 | Example 57 |
| 3 | Example 58 |
| 3 | Example 59 |
| 5 | Mix of probes from Example 58 + 59 |
| 2 | single-labelled probes (mix of 4) |
| 1 | Example 54 A-D, mix of 4 |
| 2 | Example 55 A-D, mix of 4 |
| 3 | Example 56 A-D, mix of 4 |

### Example 61

Preparation of Dendritic Complexes of the Form VSDEX: (CDx)ₙ: (Dendrimer of Example 22)ₘ; Where VSDEX is a Linear Vinylsolfone Activated Dextran of M.W. 150.000 and CDx are Antibodies; CD3, CD4, CD 19, CD23, CD33, CD34 Class I, CD56 all DAKO A/S.

General Procedure:

96 mmol of dendrimer of Example 22 and 3 mmol VSDEX were reacted in 0.28 mL 0.14 M Sodium hydrogen carbonate buffer for 6 h. at 30 C. 15 mmol antibody was then added, and the reaction was allowed to proceed for additional 18 h. Unreacted vinylsulfons were quenched by amino containing stop buffer for 1 h. The conjugates were then purified on a sepharyl S-200HR column. Relative absorbancies at 278 and 498 nm were used to determine the stoichiometries of the conjugates. Typical conjugation ratios obtained were 2-3 antibodies/DEX and 3-4 octa-fluorescein dendrimers/DEX giving an antibody:fluorescein ratio around 1:10.

### Example 62

Preparation of Dendritic Complexes of the Form VSDEX: (CDx)ₙ: (dendrimer of example 23)ₘ; where VSDEX is a linear vinylsolfone activated dextran of M.W. 150.000 and CDx are antibodies; CD3, CD4, CD 19, CD23, CD33, CD34 class I, CD56 all DAKO A/S.

General Procedure:

96 mmol of dendrimer of Example 22 and 3 mmol VSDEX were reacted in 0.28 mL 0.14 M Sodium hydrogen carbonate buffer for 6 h. at 30 C. 15 mmol antibody was then added, and the reaction was allowed to proceed for additional 18 h. Unreacted vinylsulfons were quenched by amino containing stop buffer for 1 h. The conjugates were then purified on a sepharyl S-200HR column. Relative absorbancies at 278 and 498 nm were used to determine the stoichiometries of the conjugates. Typical conjugation ratios obtained were 2-3 antibodies/DEX and 1-2 octa-fluorescein dendrimers/DEX giving an antibody:fluorescein ratio around 1:5.

### Example 63

Comparison of VSDEX: (CD3)₃: (Dendrimer of Example 22)₄ v.s. VSDEX: (CD3)₃: (Dendrimer of Example 23)₂ v.s CD23: FITC (DAKO A/S)

The 3 conjugates were tested for staining of lymphocytes by flow cytometry. VSDEX: (CD3)₃: (dendrimer of example 22)₄ gave a significantly stronger specific signal than CD23: FITC, however there was also strong unspecific staining of granylocytes.

VSDEX: (CD3)₃: (dendrimer of Example 23)₂ produced the strongest specific signal of the three conjugates, while unspecific staining of granulocytes was significantly reduced relative to the: (CD3)₃: (dendrimer of example 22)₄. Indeed this conjugate could be diluted up to 16 times, while still producing a specific signal comparable to the plain CD23: FITC conjugate.

The fact that: (CD3)₃: (dendrimer of example 23)₂ gave a stronger signal than the VSDEX: (CD3)₃: (dendrimer of Example 22)₄ despite a two fold higher fluoroscein to antibody ratio is ascribed to the increased spacing of the fluoresceins in the dendrimer of Example 23. This dendrimer features 137 bonds between closest fluoresceins, whereas the fluoresceins in the dendrimer of Example 22 are spaced by 67 bonds.

### Example 64

Preparation of Strepavidine: (Dendrimer of Example 37) Conjugate

Dendrimer of example 37, 2 mg was dissolved in water and added to 2 mg steptavidine in 0.1 mL 0.14M aqueous carbonate buffer, pH 10. After 10 min reaction the mixture was neutralized with citrate buffer to pH 7. The conjugate was purified on a sepharyl S-200HR colum, eluding after unconjugated streptavidine, but before unconjugated dendrimer.

MALDI of the assumed conjugate showed a new peak at M.W. 31500, corresponding to a steptavidine subjunit with one attached dendrimer. A peak at 13000 corresponding to unconjugated subunit was however also present, indicating that under the reaction conditions, not all subunits were labled.

### Example 65

Detection of γ-Globin mRNA Using the Dendrimer of Formula of Example 19, and Use as Signal Amplification System or Signal Enhancement System

Human cord blood was isolated and formaldehyde fixed immediately after delivery and immobilised on glass microscope slides. The slides were covered with a hybridisation solution (50 mM Tris, 10 mM NaCl, 10% w/v dextran sulphate, 30% w/v formamide, 0.1% Triton X-100®, 5 mM EDTA, pH 7.6). The hybridisation solution further comprised either the dendrimer of Example 19 (5 nM) or a peptide nucleic acid of the corresponding nucleobase sequence (40 nM), however being labelled with one fluorescein label (not a dendrimer). After 30 minutes hybridisation at 55° C., the slides were subjected to stringent washing conditions using a washing buffer containing 30% w/v formamide, 0.1% Triton X-100®, 5 mM EDTA for 30 minutes. The fluorescent signal was amplified using anti-FITC: HRP; biotinyl-tyramide; FITC-streptavidin. Following a final stringent wash, the slides were dried and mounted with Imagen Mounting Fluid (DAKO A/S). The signal obtained with the dendrimer of Example 19 was approximately 10 times stronger than the signal obtained with the single-labelled peptide nucleic acid probe. Apparently, only one peptide nucleic acid of the dendrimer binds to it's complementary mRNA sequence in the sample, the remaining 7 fluorescein-labelled peptide nucleic acids contributing to the signal amplification. It should further be noticed that the nominal concentration of peptide nucleic acid in the dendrimer-containing hybridisation buffer and control buffer containing single-labelled peptide nucleic acid is the same. Control experiments with adult male peripheral blood showed no increased background or cross-reaction of the dendrimer relative to the single-labelled peptide nucleic acid probe.

### Example 66

Use of the Dendrimers in Multi-Layer Detection Systems

The dendrimers of Example 19 and Example 21 were used. The dendrimer of Example 19 was hybridised to human cord blood as described in Example 51. Entity1 on the dendrimer of Example 21 is complementary to the 8 Entity2's on the dendrimer of Example 19. This was exploited in an extra signal-enhancing step. Following hybridisation with dendrimer of Example 19 and stringent washing, the dendrimer of Example 21 in hybridisation buffer was applied at room temperature followed by a stringent wash at 35° C. For comparison, this extra step was omitted in case of one of the slides. An amplification of about 5 times was seen when comparing the signal in case of hybridisation with the dendrimers of both Example 19 and Example 21 with the signal in case of hybridisation only with the dendrimer of Example 19. This corresponds to approximately 50 times enhancement compared to a single-labelled conventional probe (cf. Example 65).

### Example 67

Reduction of Background Signal

The experiment was carried out on metaphase chromosome spreads using the dendrimer of Example 20 and a single-labelled conventional peptide nucleic acid probe having a corresponding nucleobase sequence. The nucleobase sequence was directed at the human telomere sequence. The nucleobase sequence (TAACCC)₃. The samples were denatured in 60% formamide at 80° C. for 10 minutes. Then the dendrimer or the probe in hybridisation buffer (as defined in Example 65) was added, and the slides were allowed to cool to room temperature. Following stringent wash at 60° C., almost identical signals were observed for the dendrimer and the conventional probe, respectively, because the target is saturated even with the conventional probe. Background signal was seen in both cases. Following stringent wash at 70° C., the signal was completely absent in the case of the conventional probe, whereas the dendrimer signal was undiminished. Furthermore, the background signal in case of the dendrimer was significantly reduced by the increased washing temperature. Thus, the use a higher washing temperature leads to a higher signal-to-noise ratio.

### Example 68

Amplification System Based on Biotin:Streptavidin

The experiment was carried out as described in Example 65 using the single-labelled conventional probe of Example 65. In the final visualisation step, a commercial FITC:streptavidin conjugate was compared to plain streptavidin followed by incubation with the dendrimer of Example 16. In the conventional system, one FITC:streptavidin binds to each immobilised biotin. In the dendrimer based system, one streptavidin bind to each biotin, leaving 3 binding sites on streptavidin which can be exploited for further dendrimer binding. Accordingly, 3 dendrimers of Example 16 with a total of 24 fluorescein molecules binds to each biotin via streptavidin. A signal enhancement of approximately 15 times was observed, consistent with a 1.5 to 1 ratio of FITC to streptavidin in the commercial conjugate.

### REFERENCES

1. Wilbur et al., Bioconjugate Chem. 9, 813-825 (1998)

2. WO 98/32469 (Nycomed Imaging AS)

3. EP 271 180 (The Dow Chemical Company)

4. D. Scott Wilbur et al., Bioconjugate Chem. 9, 813-825 (1998)

5. WO 97/07398 (Dade International)

6. WO 92/20702

7. WO 92/20703

1. Dendrimer having the general formula (I)
(Entity1)ₓ₁-A-(Entity2)ₓ₂ (I)
wherein A is a dendritic core having at least one N-atomic branching point, said branching point not being part of a naturally occurring amino acid, and wherein A comprises at least one ether group,
each Entity1 and Entity2 are independently a probe, a probe substituted by one or more labelling compounds, a labelling compound, or a probe having reactive groups,
x1 and x2 are independently 0 or an integer of from 1 to 1200,
and a protected form thereof.

2. Dendrimer according to claim 1, wherein the dendritic core A extends from a single focal point through multiple generations of successive layers, each layer having one or more branching points.

3. Dendrimer according to claim 1 or 2,, wherein the dendritic core A comprises one or more of C₁₋₁₀₀ alkyl groups, C₂₋₁₀₀ alkenyl groups, C₂₋₁₀₀ alkynyl groups, said alkyl, alkenyl, and alkynyl groups optionally containing one or more functional groups and/or one or more heteroatoms, naturally or non-naturally amino acids, peptide nucleic acid moieties, LNAs, peptides, proteins, antibodies, antigens, immune complexes, DNA sequences or analogues thereof, RNA sequences or analogues thereof, macromolecules, and solid or semi-solid supports.

4. Dendrimer according to claim 3, wherein the dendritic core A comprises one or more of C₁₅₋₂₅ alkyl groups, C₁₅₋₂₅ alkenyl groups, of C₁₅₋₂₅ alkynyl groups, said alkyl, alkenyl, and alkynyl groups optionally one or more functional groups and/or one or more heteroatoms.

5. Dendrimer according to claim 3 or 4, wherein the dendritic core A comprises one or more of
—NH((CH₂)_{q}O))_{q}(CH₂)_{q}N((CH₂)_{q}C(O)—)₂,
(—CH₂)_{q}O)_{q}(CH₂)_{q}N((CH₂)_{q}O—)₂,
((—CH₂)_{q}O)_{q}(CH₂)_{q}C((CH₂)_{q}O—)₃, and
—NH((CH₂)_{q}O)_{q}(CH₂)_{q}C((CH₂)_{q}C(O)—)₃,
wherein each q independently is 0 an integer from 1 to 8, preferably from 1 to 3.

6. Dendrimer according to claims 3-5, wherein the dendritic core comprises one or more of
—NH(CH₂)₃O(CH₂)₂O(CH₂)₂O(CH₂)₃N(CH₂C(O))₂—,
—NH(CH₂)₂NHC(O)CH₂(N(CH₂CH₂)N)CH₂C(O)—,
(—NH(CH₂)₃(O)(CH₂)₂(CH₂)₂O(CH₂)₃N(CH₂COOH)CH₂C(O))_{q}, wherein q is as defined above,
—NH(CH₂)₃O(CH₂)₂O(CH₂)₂O(CH₂)₃N(COOH)CH₂C(O)—,
—NH(CH₃)₃(OCH₂CH₂)_{q}^{O(}CH₂)₃N(CH₂COOH)CH₂C(O)—, and
—NH(CH₂)₃O(CH₂)₂O(CH₂)₂O(CH₂)₃NHC(O)CH₂O(CH₂CH₂)_{q}CH₂C(O)NH—(CH₂)₃O(CH₂)₂O(CH₂)₂O(CH₂)₃N(CH₂COOH)CH₂C(O)—.

7. Dendrimer according to claims 1-6, wherein the interdistance between each Entity1 or Entity2 is at least 150 bonds.

8. Dendrimer according to claims 1-7, wherein one of the Entity1's and the Entity2's is a probe and the other is a labelling compound,
or a protected form thereof.

9. Dendrimer according to claims 1-7, wherein both of the Entity1's and Entity2's are labelling compounds or probes substituted by labelling compounds,
or a protected form thereof.

10. Dendrimer according to claim 9, wherein at least two, at least three, or at least four of the labelling compounds are different labelling compounds,
or a protected form thereof.

11. Dendrimer according to claim 9, wherein the Entity1's are a labelling compound or a probe substituted by a labelling compound, and the Entity2's are another labelling compound or a probe substituted by said another labelling compound,
or a protected form thereof.

12. Dendrimer according to claims 1-7, wherein both of the Entity1's and Entity2's are probes,
or a protected form thereof.

13. Dendrimer according to claims 1-12, wherein the labelling compound is selected from fluorophores, biotin, dinitro phenyl radical, digoxigenin, radioisotope labels, or enzyme labels, dyes, chemiluminiscence labels, hapten, antigen or antibody labels, and spin labels,
or a protected form thereof.

14. Dendrimer according to claims 1-12, wherein the probe is selected from peptide nucleic acids, RNA sequences or DNA sequences or analogues thereof, antibodies, antigens, proteins, peptides or derivatives thereof, epitopes, and biotin,
or a protected form thereof.

15. Dendrimer according to claims 1-14, wherein both x1 and x2 are 0.

16. Dendrimer according to claims 1-14, wherein x1 is 0, and x2 is an integer of from 1 to 1200.

17. Dendrimer according to claims 1-14, wherein x2 is 0, and x1 is an integer of from 1 to 1200.

18. Dendrimer according to claims 1-17, wherein the protecting group providing the protected form is selected from Fmoc, Boc, Mtt, Mmt, Dde, All, Odmab, Aloc, OtBu, OMe, OBz, Z, MOM and benzyloxycarbonyl.

19. Dendrimer according to claims 1-18, wherein x1 and x2 independently is 2^{m}, and wherein m is an integer of from 1 to 10.

20. Dendrimer according to claim 19, wherein m is 2, 3, 4 or 5.

21. Dendrimer according to claims 1-20, wherein the dendritic core A comprises one or more moieties of the structure (Ia)
Z[(Z]_{z}[)_{y}]_{z} (Ia)
wherein each Z is a group which contains at least one N-atomic branching point, said branching point not being part of a naturally occurring amino acid, and wherein the moiety of formula (Ia) comprises at least one ether group, and wherein each y independently is 2 or 3, and z is an integer of from 1 to 10,
with the proviso that y^{z}≦1200,
and a protected form thereof.

22. Dendrimer according to claim 21, wherein the dendritic core A comprises at least two moieties of the structure (Ia) connected to a naturally or non-naturally amino acid, a peptide nucleic acid moiety, an LNA, a peptide, a protein, an antibody, an antigen, an immune complex, a DNA sequence or an analogue thereof, a RNA sequence or an analogue thereof, a macromolecule, and/or a solid or semi-solid support.

23. Dendrimer according to claim 21 or 22, wherein each Z comprises one or more of C₁₋₁₀₀ alkyl groups, C₂₋₁₀₀ alkenyl groups, C₂₋₁₀₀ alkynyl groups, said alkyl, alkenyl, and alkynyl groups optionally containing one or more functional groups and/or one or more heteroatoms, naturally or non-naturally amino acids, peptide nucleic acid moieties, LNAs, peptides, proteins, antibodies, antigens, immune complexes, DNA sequences and analogues thereof, RNA sequences and analogues thereof, and macromolecules.

24. Dendrimer according to claim 23, wherein the dendritic core A comprises one or more of C₁₅₋₂₅ alkyl groups, C₁₅₋₂₅ alkenyl groups, of C₁₅₋₂₅ alkynyl groups, said alkyl, alkenyl, and alkynyl groups optionally one or more functional groups and/or one or more heteroatoms.

25. Dendrimer according to claim 23 or 24, wherein the dendritic core A comprises one or more of
—NH((CH₂)_{q}O))_{q}(CH₂)_{q}N((CH₂)_{q}C(O)—)₂,
((—CH₂)_{q}O)_{q}(CH₂)_{q}N((CH₂)_{q}O—)₂,
((—CH₂)_{q}O)_{q}(CH₂)_{q}C((CH₂)_{q}O)₃, and
—NH((CH₂)_{q}O)_{q}(CH₂)_{q}C((CH₂)_{q}C(O)—)₃,
wherein each q independently is 0 an integer from 1 to 8, preferably from 1 to 3.

26. Dendrimer according to claims 21-26, wherein the dendritic core A comprises one or more of
—NH(CH₂)₃O(CH₂)₂O(CH₂)₂O(CH₂)₃N(CH₂C(O))₂—,
—NH(CH₂)₂NHC(O)CH₂(N(CH₂CH₂)N)CH₂C(O)—(—NH(CH₂)₃(O)(CH₂)₂O(CH₂)₂O(CH₂)₃N(CH₂COOH)CH₂C(O))_{q}, wherein q is as defined above,
—NH(CH₂)₃O(CH₂)₂(CH₂)₂O(CH₂)₃N(COOH)CH₂C(O)—,
—NH(CH₃)₃(OCH₂CH₂)_{q}O(CH₂)₃N(CH₂COOH)CH₂C(O)—, and
—NH(CH₂)₃O(CH₂)₂O(CH₂)₂O(CH₂)₃NHC(O)CH₂O(CH₂CH₂)_{q}CH₂C(O)NH—(CH₂)₃O(CH₂)₂O(CH₂)₂O(CH₂)₃N(CH₂COOH)CH₂C(O)—.

27. Dendrimer according to claims 21-26, wherein the interdistance between each Entity1 or Entity2 is at least 150 bonds.

28. Dendrimer according to claims 21-27, wherein one of the Entity1's and the Entity2's is a probe and the other is a labelling compound,
or a protected form thereof.

29. Dendrimer according to claims 21-27, wherein both of the Entity1's and Entity2's are labelling compounds or probes substituted by labelling compounds,
or a protected form thereof.

30. Dendrimer according to claim 29, wherein at least two, at least three, or at least four of the labelling compounds are different labelling compounds,
or a protected form thereof.

31. Dendrimer according to claim 29, wherein the Entity1's are a labelling compound or a probe substituted by a labelling compound, and the Entity2's are another labelling compound or a probe substituted by said another labelling compound,
or a protected form thereof.

32. Dendrimer according to claims 21-27, wherein both of the Entity1's and the Entity2's are probes,
or a protected form thereof.

33. Dendrimer according to claims 21-32, wherein the labelling compound is selected from fluorophores, biotin, dinitro phenyl radical, digoxigenin, radioisotope labels, or enzyme labels, dyes, chemiluminiscence labels, hapten, antigen or antibody labels, and spin labels,
or a protected form thereof.

34. Dendrimer according to claims 21-32, wherein the probe is selected from peptide nucleic acids, RNA sequences or DNA sequences or analogues thereof, antibodies, antigens, proteins, peptides or derivatives thereof, epitopes, and biotin,
or a protected form thereof.

35. Dendrimer according to claims 21-34, wherein both x1 and x2 are 0.

36. Dendrimer according to claims 21-34, wherein x1 is 0, and x2 is an integer of from 1 to 1200.

37. Dendrimer according to claims 21-34, wherein x2 is 0, and x1 is an integer of from 1 to 1200.

38. Dendrimer according to claims 21-37, wherein x1 and x2 independently is 2ₘ, and wherein m is an integer of from 1 to 10.

39. Dendrimer according to claim 38, wherein m is 2, 3, 4 or 5.

40. Dendrimer according to claims 1-39, wherein at least one of the Entity1's and the Entity2's is connected to a naturally or non-naturally amino acid terminally or internally, a peptide nucleic acid terminally or internally, a peptide terminally or internally, an LNA terminally or internally, a protein terminally or internally, an antibody, an antigen, an immune complex, a RNA sequence or an analogues thereof terminally or internally, a DNA sequence or an analogue thereof terminally or internally, a macromolecule terminally or internally, or a solid or semi-solid support.

41. Protected dendrimer according to claims 1-40.

42. Dendrimer complex comprising at least one dendrimer according to claims 1-41 connected to a naturally or non-naturally amino acid terminally or internally, a peptide nucleic acid terminally or internally, an LNA terminally or internally, a peptide terminally or internally, a protein terminally or internally, an antibody, an antigen, an immune complex, a RNA sequence or an analogue thereof terminally or internally, a DNA sequence or an analogue terminally or internally, a macromolecule terminally or internally, or a solid or semi-solid support,
or a protected form thereof.

43. Dendrimer complex according to claim 42, wherein the dendrimer is connected via at least one of the Entity1's and/or the Entity2's.

44. Dendrimer complex according to claim 42, wherein the dendrimer is connected via a group or groups of the dendritic core A.

45. Dendrimer complex according to claims 42-44, wherein the dendrimer complex comprises 1-50 dendrimers according to claims 1-41.

46. Dendrimer complex according to claim 45, wherein the dendrimer complex comprises 1-35, 1-30, 1-25, 1-15, 1-10, 1-8,1-5, or 1-3 dendrimers according to claims 1-41.

47. Dendrimer complex according to claims 42-46, wherein the dendrimer is internally connected to a naturally or non-naturally occurring amino acid, a peptide nucleic acid, an LNA, a protein, a RNA sequence or a DNA sequence, a macromolecule, or a solid or a semi-solid support.

48. Protected dendrimer complex according to claims 42-47.

49. Use of a dendrimer according to claims 1-41 or a dendrimer complex according to claims 42-48 for detecting the presence of nucleic acid sequences, antibodies, antigens, immune complexes, proteins, or peptides in a sample.

50. A detection system comprising at least one dendrimer according to claims 1-41 and/or at least one dendrimer complex according to claims 42-48.

51. A signal amplification system comprising at least one dendrimer according to claims 1-41 and/or at least one dendrimer complex according to claims 42-48.

52. Use of a dendrimer according to claims 1-41 or a dendrimer complex according to claims 42-48 as a detection system.

53. Use of a dendrimer according to claims 1-41 or a dendrimer complex according to claims 42-48 as a signal amplification system.

54. Use of the detection system according to claim 50 or the amplification system according to claim 51 for detecting the presence of nucleic acid sequences, antibodies, antigens, immune complexes, proteins, or peptides in a sample.

55. Use according to claim 54, wherein the sample is a blood sample, a bone marrow sample, a chromosome spread, a tissue sample, a tissue section, a cell smear, a biopsy, an organ, a swap, a suspension of cells or parts thereof, or a whole cell or parts thereof.

56. Use of a dendrimer according to claims 1-41 or a dendrimer complex according to claims 42-48 in an in vitro diagnostic method or an in vivo diagnostic method.

57. Use of a dendrimer according to claims 1-41 or a dendrimer according to claims 42-48 for labelling a compound.

58. Use according to claim 57, wherein the compound to be labelled is selected from peptide nucleic acids, LNAs, RNA sequences, DNA sequences or analogues thereof, antibodies, antigens, proteins, peptides or derivatives thereof, epitopes, streptavidin, and biotin.

59. Use according to claims 57 or 58, wherein the dendrimer or dendrimer complex comprises one or more labelling compounds selected from fluorophores, biotin, dinitro phenyl radicals, digoxigenin, radioisotope labels, enzyme labels, dyes, chemiluminiscence labels, haptens, antigen or antibody labels, and spin labels.

60. Labelling kit for labelling a compound comprising one or more dendrimers according to claims 1-41 and/or dendrimer complexes according to claims 42-48.

61. Labelling kit according to claim 60, further comprising a labelling compound.

62. Labelling kit according to claim 60, wherein the dendrimer or dendrimer complex comprises a labelling compound.

63. Labelling kit according to claim 60 or 61, wherein the labelling compound is selected from fluorophores, biotin, dinitro phenyl radicals, digoxigenin, radioisotope labels, enzyme labels, dyes, chemiluminiscence labels, haptens, antigen or antibody labels, and spin labels.

64. Detection kit for detecting a compound comprising one or more dendrimers according to claims 1-42 and/or a dendrimer complex according to claims 42-48, wherein the dendrimer and/or the dendrimer complex comprise(s) one or more labelling compounds.

65. Detection kit according to claim 64, wherein the labelling compound is selected from fluorophores, biotin, dinitro phenyl radicals, digoxigenin, radioisotope labels, enzyme labels, dyes, chemiluminiscence labels, haptens, antigen or antibody labels, and spin labels.

66. A process for preparing a dendrimer having the general formula (I)
(Entity1)ₓ₁-A-(Entity2)ₓ₂ (I)
wherein A is a dendritic core having at least one N-atomic branching point, said branching point not being part of a naturally occurring amino acid, and wherein A comprises at least one ether group,
each Entity1 and Entity2 are independently probes which optionally are substituted by one or more labelling compounds, a labelling compound, or a probe having reactive groups,
x1 and x2 are independently 0 or an integer of from 1 to 1200,
or a protected form thereof
comprising
preparing a dendritic core A by chemical/peptide synthesis, depending on the components of the dendritic core A, which dendritic core A has a number of protecting groups, which can be removed by deprotection,
if desired, removing the protecting groups by use of a suitable deprotecting agent,
if desired, coupling the Entity1's and/or the Entity2's to the dendritic core A, thereby obtaining the dendrimer or the protected form thereof.

67. A process for preparing a dendrimer having the general formula (I)
(Entity1)ₓ₁-A-(Entity2)ₓ₂ (I)
wherein A is a dendritic core having at least one N-atomic branching point, said branching point not being part of a naturally occurring amino acid, and wherein A comprises at least one ether group,
each Entity1 and Entity2 are independently probes which optionally are substituted by one or more labelling compounds, a labelling compound, or a probe having reactive groups,
x1 and x2 are independently 0 or an integer of from 1 to 1200,
or a protected form thereof,
comprising
preparing building blocks for the dendritic core A by chemical/peptide synthesis, depending on the components of the dendritic core A,
assembling the dendritic core A, which dendritic core A has a number of protecting groups, which can be removed by deprotection,
if desired, removing the protecting groups by use of a suitable deprotecting agent,
if desired, coupling the Entity1's and/or the Entity2's to the dendritic core A, thereby obtaining the dendrimer or the protected form thereof.

68. Dendrimer obtainable by the process according to claim 66 or 67.
